(19) United States
(12) Patent Application Publication
(10) Pub. No.: US 2009/0042233 A1
(43) Pub. Date: Feb. 12, 2009

(54) NOVEL PROTEIN MEMBER OF THE RAS/MAPK PATHWAY, ANTIBODIES THEREOF AND METHODS AND KITS OF USING SAME

(76) Inventors: Marc THERRIEN, Montreal-West (CA); Malha Sahmi, Ville Lemoyne (CA); Melanie Douziech, St-Jean-sur-Richelieu (CA)

Correspondence Address: GOUDREAU GAGE DUBUC, 2000 MCGILL COLLEGE, SUITE 2200, MONTREAL, QC, H3A 3H3, CA

(21) Appl. No.: 11/695,254

(22) Filed: Apr. 2, 2007

Related U.S. Application Data
(60) Provisional application No. 60/787,630, filed on Mar. 31, 2006.

(51) Int. Cl. C12Q 1/02; C07K 14/00; C07K 16/00; G01N 33/53; C07K 1/14; C12N 9/12; C07H 21/00; C07H 21/02; C12N 5/00

(52) U.S. Cl. 435/29; 530/350; 530/387.9; 436/501; 530/413; 536/23.1; 536/24.3; 435/375; 435/194

(57) ABSTRACT

A polypeptide comprising a SAM domain, the sequence of said SAM domain being as set forth in SEQ ID NO: 33.

### CROSS REFERENCE TO RELATED APPLICATIONS

This application claims priority on U.S. provisional application No. 60/744,090, filed on 31 Mar. 2006 and this document is incorporated herein in its entirety by reference.

### FIELD OF THE INVENTION

The present invention relates to a novel protein member of the RAS/MAPK pathway, antibodies thereof and methods and kits of using same. More specifically, the present invention is concerned with a novel SAM domain-containing polypeptide.

### BACKGROUND OF THE INVENTION

Signal transmission via the RAF/MEK/ERK pathway, also known as the MAPK module, is a central event triggered by the small GTPase RAS to regulate a number of basic cellular processes in metazoans, including cell proliferation, differentiation and survival (Pearson et al., 2001). Unrestrained signaling through this pathway caused for instance by activating mutations in specific isoforms of either RAS or RAF, has been linked to several types of cancer in humans and, for some of these, at an impressively high frequency (Malumbres and Barbacid, 2003; Wellbrock et al., 2004). Because of potential benefits to human health, extensive efforts have been devoted to describe in molecular terms the signal transfer mechanism within the RAS/MAPK pathway. Despite significant progress, a number of events have proven particularly challenging. One notable example is the mechanism leading to the activation of the RAF serine/threonine kinase.

Three RAF members have been identified in mammals (A-, B- and C-RAF/Raf-1) and homologues are present in other metazoans, including C. elegans and Drosophila where a single gene encoding a protein more closely related to B-RAF has been identified (Chong et al., 2003; Dhillon and Kolch, 2002). RAF proteins comprise an N-terminal regulatory region followed by a C-terminal catalytic domain. The N-terminal region includes a RAS-binding domain (RBD), a cysteine-rich domain (CRD) and an inhibitory 14-3-3-binding site encompassing serine 259 (S259) in C-RAF. The binding of 14-3-3 to this latter site requires the phosphorylation of the S259-like residue in RAF proteins, which in turn mediates their cytoplasmic retention in unstimulated cells (Morrison and Cutler, 1997). Upon RTK-dependent activation, GTP-loaded RAS binds the RBD of RAF and facilitates the dephosphorylation of the S259-like residue, thereby releasing 14-3-3 and promoting the association of RAF to the membrane (Dhillon et al., 2002; Jaumot and Hancock, 2001; Light et al., 2002). A number of phosphorylation events are then required to fully induce RAF catalytic activity (Chong et al., 2003). Although some are isozyme-specific, two are probably common to all members and affect conserved serine/threonine residues (T599 and S602 in B-RAF) situated in the activation loop of the kinase domain (Zhang and Guan, 2000). Mutational analyses as well as a recent crystallographic study of the B-RAF kinase domain strongly suggest that phosphorylation of these residues plays a critical role in the final stage of activation by destabilizing an inhibitory interaction that takes place between the P loop (sub-domain 1) and the DFG motif (sub-domain VII)/activation loop of the kinase domain (Wan et al., 2004). The mechanism and kinase(s) leading to the phosphorylation of these residues are unknown.

A number of scaffold proteins have also been suggested to regulate RAF activity (Kolch, 2000). However, their mode of action and functional interdependency is poorly understood. One example corresponds to the Kinase Suppressor of RAS (KSR) members, which are known to assemble RAF, MEK and MAPK into functional complexes (Morrison and Davis, 2003). Interestingly, these proteins are structurally related to RAF, although they have some key differences. For instance, they do not contain an RBD, but comprise a conserved region called CA1 that was found in Drosophila to mediate an interaction between KSR and RAF (Roy et al., 2002). Further, they possess a kinase-like domain that constitutively binds MEK, but which appears to be devoid of kinase activity (Morrison and Davis, 2003). While the function of KSR as a scaffold of the MAPK module has been convincingly documented, genetic and biochemical characterization of the single Drosophila KSR isoform suggested that its activity is also required upstream of RAF (Anselmo et al., 2002; Therrien et al., 1995). This other role, however, has not been determined.

Connector eNhancer of KSR(CNK) is another scaffold protein acting as a putative regulator of RAF activity. As for KSR, its activity is essential for multiple receptor tyrosine kinase (RTK) signaling events where it appears to regulate the MAPK module at the level of RAF (Therrien et al., 1998). CNK homologues have been identified in other metazoans and evidence gathered in mammalian cell lines supports their participation in the regulation of B- and C-RAF (Bumeister et al., 2004; Lanigan et al., 2003; Ziogas et al., 2005). A similar conclusion was also recently reached in C. elegans (Rocheleau et al., 2005). In flies, CNK associates directly with the catalytic domain of RAF through a short amino acid sequence called the RAF-interacting Motif (RIM) and modulates RAF activity according to the RTK signaling status (Douziech et al., 2003; Laberge et al., 2005). In absence of RTK signals, CNK-bound RAF is inhibited by a second motif adjacent to the RIM, called the Inhibitory Sequence (IS). In contrast, upon RTK activation, CNK integrates RAS and Src activity which in turn leads to RAF activation. The ability of RAS to promote RAF activation was found to strictly depend on two domains: a Sterile-Alpha Motif (SAM) domain and the so-called Conserved Region in CNK (CRIC) located in the N-terminal region of CNK (Douziech et al., 2003). The molecular role of these domains is currently unknown. On the other hand, the binding of a Src family kinase, Src42, to an RTK-dependent phospho-tyrosine residue (pY1163) located C-terminal to the IS motif appears to release the inhibitory effect that the IS motif imposes on RAF catalytic function (Laberge et al., 2005).

The present description refers to a number of documents, the content of which is herein incorporated by reference in their entirety.

### SUMMARY OF THE INVENTION

The present invention relates to the identification of a novel and evolutionarily conserved SAM domain-containing protein, Hyphen. It also identified the role of the Sterile Alpha Motif (SAM) and Conserved Region in CNK (CRIC) domains of Connector eNhancer of KSR(CNK) during RAS-dependent RAF activation. Strikingly, the present invention shows that their activity is mediated by Kinase Suppressor of RAS (KSR), and that KSR stimulates RAF catalytic function independently of its capacity to bridge RAF and MEK. This effect occurs at a step upstream of the activation loop phosphorylation, but downstream of the dephosphorylation of the S259-like residue, thus indicating that it regulates the final stage of RAF activation. While the catalytically devoid KSR kinase domain appears to be the primary effector of this event, CNK participates in at least two ways: 1) it assembles a KSR/RAF complex in vivo by interacting separately with the kinase domains of KSR and RAF through its SAM domain and RIM element, respectively; 2) its CRIC region promotes CNK-bound KSR activity toward RAF in a RAS-dependent manner. The present invention shows that the KSR/CNK interaction depends on Hyphen, whose presence is essential for RAS-induced signaling through the MAPK module at a step upstream of RAF. Together, the present invention unveils a network of interacting scaffolds that regulates the RAS-dependent catalytic function of RAF.

### DEFINITIONS

Unless defined otherwise, the scientific and technological terms and nomenclature used herein have the same meaning as commonly understood by a person of ordinary skill to which this invention pertains. Commonly understood definitions of molecular biology terms can be found for example in Dictionary of Microbiology and Molecular Biology, 2nd ed. (Singleton et al., 1994, John Wiley & Sons, New York, N.Y.), The Harper Collins Dictionary of Biology (Hale & Marham, 1991, Harper Perennial, New York, N.Y.), Rieger et al., Glossary of genetics: Classical and molecular, 5th edition, Springer-Verlag, New-York, 1991; Alberts et al., Molecular Biology of the Cell, 4th edition, Garland science, New-York, 2002; and, Lewin, Genes VII, Oxford University Press, New-York, 2000. Generally, the methods traditionally used in molecular biology, such as preparative extractions of plasmid DNA, centrifugation of plasmid DNA in cesium chloride gradient, agarose or acrylamide gel electrophoresis, purification of DNA fragments by electroelution, phenol or phenol-chloroform extraction of proteins, ethanol or isopropanol precipitation of DNA in saline medium, transformation into bacteria or transfection into cells, procedure for cell culture, infection, methods and the like are common methods used in the art. Such standard techniques can be found in reference manuals such as for example Sambrook et al. (2000, Molecular Cloning—A Laboratory Manual, Third Edition, Cold Spring Harbor Laboratories); and Ausubel et al. (1994, Current Protocols in Molecular Biology, John Wiley & Sons, New-York). In addition, methods and procedures to produce transgenic animals are well-known in the art and described in details for example in: Hogan et al., 1994, Manipulating the Mouse Embryo, Cold Spring Harbor Laboratory Press; Nagy et al., 2002, Manipulating the Mouse Embryo, 3rd edition, Cold Spring Harbor Laboratory Press.

The use of the word “a” or “an” when used in conjunction with the term “comprising” in the claims and/or the specification may mean “one” but it is also consistent with the meaning of “one or more”, “at least one”, and “one or more than one”.

Throughout this application, the term “about” is used to indicate that a value includes the standard deviation of error for the device or method being employed to determine the value. In general, the terminology “about” is meant to designate a possible variation of up to 10%. Therefore, a variation of 1, 2, 3, 4, 5, 6, 7, 8, 9 and 10% of a value is included in the term about.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

Nucleotide sequences are presented herein by single strand, in the 5′ to 3′ direction, from left to right, using the one-letter nucleotide symbols as commonly used in the art and in accordance with the recommendations of the IUPAC IUB Biochemical Nomenclature Commission.

As used herein, “nucleic acid molecule” or “polynucleotides”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or synthesized. DNA can be double-stranded or single-stranded (coding strand or non-coding strand [antisense]). Conventional ribonucleic acid (RNA) and deoxyribonucleic acid (DNA) are included in the terms “nucleic acid” and “polynucleotides” as are analogs thereof. A nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Int'l Pub. No. WO 95/32305), phosphorothioate linkages, methylphosphonate linkages or combinations thereof. Sugar moieties of the nucleic acid may be ribose or deoxyribose, or similar compounds having known substitutions, e.g., 2′ methoxy substitutions (containing a 2′-O-methylribofuranosyl moiety; see PCT No. WO 98/02582) and/or 2′ halide substitutions. Nitrogenous bases may be conventional bases (A, G, C, T, U), known analogs thereof (e.g., inosine or others; see The Biochemistry of the Nucleic Acids 5-36, Adams et al., ed., 11th ed., 1992), or known derivatives of purine or pyrimidine bases (see, Cook, PCT Int'l Pub. No. WO 93/13121) or “a basic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs).

As used herein, “protein” or “polypeptide” means any peptide-linked chain of amino acids, regardless of post-translational modifications (e.g. acetylation, phosphorylation, glycosylation, sulfatation, sumoylation, prenylation, ubiquitination, etc). A “HYP protein” or a “HYP polypeptide” is an expression product of HYP nucleic acid (e.g. HYP gene) such as native human HYP protein (FIG. 13), a HYP natural splice variant, a HYP allelic variant or a HYP protein homolog (e.g. mouse HYP, FIGS. 10, 12 and 13) that shares at least 60% (but preferably, at least 65, 70, 75, 80, 85, 86, 87, 88, 89, 90, 91, 92, 93, 94, 95, 96, 97, 98, 99, 100%) amino acid sequence identity with HYP and displays functional activity of native HYP protein. For the sake of brevity, the units (e.g. 66, 67 . . . 81, 82% . . . ) have not been specifically recited but are nevertheless considered within the scope of the present invention.

Nucleic acid sequences may be detected by using hybridization with a complementary sequence (e.g., oligonucleotide probes—see U.S. Pat. Nos. 5,503,980 (Cantor); 5,202,231 (Drmanac et al.); 5,149,625 (Church et al.); 5,112,736 (Caldwell et al.); 5,068,176 (Vijg et al.); and 5,002,867 (Macevicz)). Hybridization detection methods may use an array of probes (e.g., on a DNA chip) to provide sequence information about the target nucleic acid which selectively hybridizes to an exactly complementary probe sequence in a set of four related probe sequences that differ by one nucleotide (see U.S. Pat. Nos. 5,837,832 and 5,861,242 (Chee et al.). In addition, any other well-known hybridization technique (Northern blot, dot blot, Southern blot) may be used in accordance with the present invention.

Nucleic Acid Hybridization. Nucleic acid hybridization depends on the principle that two single-stranded nucleic acid molecules that have complementary base sequences will reform the thermodynamically favored double-stranded structure if they are mixed under the proper conditions. The double-stranded structure will be formed between two complementary single-stranded nucleic acids even if one is immobilized on a nitrocellulose filter. In the Southern or Northern hybridization procedures, the latter situation occurs. The DNA/RNA of the individual to be tested may be digested with a restriction endonuclease if applicable, prior to its fractionation by agarose gel electrophoresis, conversion to the single-stranded form, and transfer to nitrocellulose paper, making it available for reannealing to the hybridization probe. Non-limiting examples of hybridization conditions can be found in Ausubel, F. M. et al., Current protocols in Molecular Biology, John Wiley & Sons, Inc., New York, N.Y. (1994). For purposes of illustration, an example of moderately stringent conditions for testing the hybridization of a polynucleotide of the present invention with other polynucleotides, include prewashing, in a solution of 5×SSC, 0.5% SDS, 1 mM EDTA (pH 8.0); hybridizing at 50° C.-60° C., 5×SSC and 100 μg/ml denatured salmon sperm DNA overnight (12-16 hours); followed by washing twice at 60° C. for 15 minutes with each of 2×SSC, 0.5×SSC and 0.2×SSC containing 0.1% SDS. For example for highly stringent hybridization conditions, the hybridization temperature is changed to 62, 63, 64, 65, 66, 67 or 68° C. One skilled in the art will understand that the stringency of hybridization can be readily manipulated, such as by altering the salt and SDS concentration of the hybridizing and washing solutions and/or temperature at which the hybridization is performed. The temperature and salt concentration selected is determined based on the melting temperature (Tm) of the DNA hybrid. Other protocols or commercially available hybridization kits using different annealing and washing solutions can also be used as well known in the art. The use of formamide in different mixtures to lower the melting temperature may also be used and is well known in the art.

A “probe” is meant to include a nucleic acid oligomer that hybridizes specifically to a target sequence in a nucleic acid or its complement, under conditions that promote hybridization, thereby allowing detection of the target sequence or its amplified nucleic acid. Detection may either be direct (i.e., resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.”

By “sufficiently complementary” is meant a contiguous nucleic acid base sequence that is capable of hybridizing to another sequence by hydrogen bonding between a series of complementary bases. Complementary base sequences may be complementary at each position in sequence by using standard base pairing (e.g., G:C, A:T or A:U pairing) non standard base pairing (e.g., I:C) or may contain one or more residues (including a basic residues) that are not complementary by using standard base pairing, but which allow the entire sequence to specifically hybridize with another base sequence in appropriate hybridization conditions. Contiguous bases of an oligomer are preferably at least about 80% (81, 82, 83, 84, 85, 86, 87, 88, 89, 90, 91, 92, 93, 94, 95, 96, 97, 98, 99, 100%), more preferably at least about 90% complementary to the sequence to which the oligomer specifically hybridizes. In reference to more specific nucleic acid molecules of the present invention, the binding free energy for a nucleic acid molecule with its complementary sequence is sufficient to allow the relevant function of the nucleic acid to proceed (e.g., RNAi activity). For example, the degree of complementarity between the sense and antisense region (or strand) of the siRNA construct can be the same or can be different from the degree of complementarity between the antisense region of the siRNA and the target RNA sequence (e.g., HYP or ERT RNA sequence). Complementarity to the target sequence of less than 100% in the antisense strand of the siRNA duplex (including deletions, insertions and point mutations) is reported to be tolerated when these differences are located between the 5′-end and the middle of the antisense siRNA (Elbashir et al., 2001, EMBO, 20(23):68-77-6888). Determination of binding free energies for nucleic acid molecules is well known in the art (e.g., see Turner et al., 1987, J. Am. Chem. Soc. 190:3783-3785; Frier et al., 1986 Proc. Nat. Acad. Sci. USA, 83: 9373-9377) “Perfectly complementary” means that all the contiguous residues of a nucleic acid molecule will hydrogen bond with the same number of contiguous residues in a second nucleic acid sequence. Appropriate hybridization conditions are well known to those skilled in the art, can be predicted readily based on sequence composition and conditions, or can be determined empirically by using routine testing (see Sambrook et al., (cf. Molecular Cloning: A Laboratory Manual, Third Edition, edited by Cold Spring Harbor Laboratory, 2000) at §§ 1.90-1.91, 7.37-7.57, 9.47-9.51 and 11.47-11.57, particularly at §§ 9.50-9.51, 11.12-11.13, 11.45-11.47 and 11.55-11.57). Sequences that are “sufficiently complementary” allow stable hybridization of a probe sequence to a target sequence, even if the two sequences are not completely identical.

A detection step may use any of a variety of known methods to detect the presence of nucleic acid by hybridization to a probe oligonucleotide. One specific example of a detection step uses a homogeneous detection method such as described in detail previously in Arnold et al. Clinical Chemistry 35:1588-1594 (1989), and U.S. Pat. Nos. 5,658,737 (Nelson et al.), and 5,118,801 and 5,312,728 (Lizardi et al.).

The types of detection methods in which probes can be used include Southern blots (DNA detection), dot or slot blots (DNA, RNA), and Northern blots (RNA detection). Labeled proteins could also be used to detect a particular nucleic acid sequence to which it binds (e.g. protein detection by far western technology: Guichet et al., 1997, Nature 385(6616): 548-552; and Schwartz et al., 2001, EMBO 20(3): 510-519). Other detection methods include kits containing reagents of the present invention on a dipstick setup and the like. Of course, it might be preferable to use a detection method which is amenable to automation. A non-limiting example thereof includes a chip or other support comprising one or more (e.g. an array) different probes.

As used herein, the term “ligand” broadly to refer to natural, synthetic or semi-synthetic molecules. The term “molecule” therefore denotes for example chemicals, macromolecules, cell or tissue extracts (from plants or animals) and the like. Non limiting examples of molecules include nucleic acid molecules, peptides, antibodies, carbohydrates and pharmaceutical agents. The ligand appropriate for the present invention can be selected and screened by a variety of means including random screening, rational selection and by rational design using for example protein or ligand modeling methods such as computer modeling. The terms “rationally selected” or “rationally designed” are meant to define compounds which have been chosen based on the configuration of interacting domains of the present invention. As will be understood by the person of ordinary skill, macromolecules having non-naturally occurring modifications are also within the scope of the term “ligand”. For example, peptidomimetics, well known in the pharmaceutical industry and generally referred to as peptide analogs can be generated by modeling as mentioned above. Similarly, in a preferred embodiment, the polypeptides of the present invention are modified to enhance their stability. It should be understood that in most cases this modification should not alter the biological activity of the interaction domain.

As used herein the terms “Hyphen protein” or “HYP protein” are used interchangeably herein to encompass for simplicity sake, unless otherwise qualified, not only Drosophila Hyphen but any other species equivalent including, without being so limited, those disclosed in FIGS. 12 and 13.

As used herein, the term “purified” in the expression “purified antibody” is simply meant to distinguish man-made antibody from an antibody that may naturally be produced by an animal against its own antigens. Hence, raw serum and hybridoma culture medium containing anti-hyphen antibody are “purified antibodies” within the meaning of the present invention.

As used herein, the term “anti-Hyphen antibody” or “immunologically specific anti-Hyphen antibody” refers to an antibody that specifically binds to (interacts with) a Hyphen protein and displays no substantial binding to other naturally occurring proteins other than the ones sharing the same antigenic determinants as the Hyphen protein. The term antibody or immunoglobulin is used in the broadest sense, and covers monoclonal antibodies (including full length monoclonal antibodies), polyclonal antibodies, multispecific antibodies, and antibody fragments so long as they exhibit the desired biological activity. Antibody fragments comprise a portion of a full length antibody, generally an antigen binding or variable region thereof. Examples of antibody fragments include Fab, Fab′, F(ab′)₂, and Fv fragments, diabodies, linear antibodies, single-chain antibody molecules, single domain antibodies (e.g., from camelids), shark NAR single domain antibodies, and multispecific antibodies formed from antibody fragments. Antibody fragments can also refer to binding moieties comprising CDRs or antigen binding domains including, but not limited to, VH regions (V_{H}, V_{H}—V_{H}), anticalins, PepBodies™, antibody-T-cell epitope fusions (Troybodies) or Peptibodies. Additionally, any secondary antibodies, either monoclonal or polyclonal, directed to the first antibodies would also be included within the scope of this invention.

A monoclonal antibody refers to an antibody obtained from a population of substantially homogeneous antibodies, e.g., the individual antibodies comprising the population are identical except for possible naturally occurring mutations that may be present in minor amounts. Monoclonal antibodies are highly specific, being directed against a single antigenic site. Furthermore, in contrast to conventional (e.g., polyclonal) antibody preparations which typically include different antibodies directed against different determinants (e.g., epitopes) on an antigen, each monoclonal antibody is directed against at least a single determinant on the antigen. The modifier “monoclonal” indicates the character of the antibody as being obtained from a substantially homogeneous population of antibodies, and is not to be construed as requiring production of the antibody by any particular method. For example, monoclonal antibodies may be made by the hybridoma method first described by Kohler et al., Nature 256:495 (1975), or may be made by recombinant DNA methods (see, e.g., U.S. Pat. No. 4,816,567). Monoclonal antibodies may also be isolated from phage antibody libraries, for example, using the techniques described in Clackson et al., Nature 352:624-628 (1991) and Marks et al., J. Mol. Biol. 222:581-597 (1991). Monoclonal antibodies can also be isolated using the techniques described in U.S. Pat. Nos. 6,025,155 and 6,077,677 as well as U.S. Patent Application Publication Nos. 2002/0160970 and 2003/0083293 (see also, e.g., Lindenbaum, et al., Nucleic Acids Research 32 (21):0177 (2004)).

Monoclonal antibodies can include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81: 6851-6855 (1984) for mouse-human chimeric antibodies).

A hypervariable region refers to the amino acid residues of an antibody which are responsible for antigen-binding. The hypervariable region comprises amino acid residues from a complementarity determining region or CDR (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a hypervariable loop (e.g., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196: 901-917 (1987)). Framework or FR residues are those variable domain residues other than the hypervariable region residues. For antibodies described herein, the CDR and framework regions are identified based on the Kabat numbering system except that the CDR1 of the heavy chain is defined by Oxford Molecular's AbM definition as spanning residues 26 to 35. The Oxford Molecular's AbM antibody modeling software (http://people.cryst.cck.ac.uk/˜ubc07s/) (Martin et al., Proc. Natl. Acad. Sci. USA, 86, 9268-9272 (1989); Martin et al., Methods Enzymol., 203, 121-153 (1991); Pedersen et al., Immunomethods, 1, 126 (1992); and Rees et al., In Sternberg M. J. E. (ed.), Protein Structure Prediction. Oxford University Press, Oxford, 141-172. (1996)) combines the Kabat CDR and the Chothia hypervariable region numbering systems to define CDRs.

Humanized forms of non-human (e.g., murine) antibodies may be chimeric antibodies which contain minimal sequence derived from non-human immunoglobulin. For the most part, humanized antibodies are human immunoglobulins (recipient or acceptor antibody) in which hypervariable region residues of the recipient are replaced by hypervariable region residues from a non-human species (donor antibody) such as mouse, rat, rabbit or nonhuman primate having the desired specificity, affinity, and capacity. In addition, individual or groups of Fv framework region (FR) residues of the human immunoglobulin may be replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable regions or domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (e.g., Fc), typically that of a human immunoglobulin (see, e.g., Queen et al., Proc. Natl. Acad. Sci. USA 86:10029 (1989), and Foote and Winter, J. Mol. Biol. 224: 487 (1992)).

Single-chain Fv or scFv antibody fragments may comprise the V_{H} and V_{L} regions or domains of antibody, wherein these domains are present in a single polypeptide chain. Generally, the Fv polypeptide further comprises a polypeptide linker between the V_{H} and V_{L} domains which enables the scFv to form the desired structure for antigen binding (for a review, see, e.g., Pluckthun in The Pharmacology of Monoclonal Antibodies, vol. 113, Rosenburg and Moore eds. Springer-Verlag, New York, pp. 269-315 (1994)).

Diabody refers to small antibody fragments with two antigen-binding sites, which fragments comprise a heavy chain variable domain (V_{H}) connected to a light chain variable domain (V_{L}) in the same polypeptide chain (V_{H}—V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90: 6444-6448 (1993).

Linear antibody refers to antibodies such as those described in Zapata et al., Protein Eng. 8(10): 1057-1062 (1995). Briefly, these antibodies comprise a pair of tandem Fd segments (V_{H}—C_{H}1-V_{H}—C_{H}1) which form a pair of antigen binding regions. Linear antibodies can be bispecific or monospecific.

An isolated antibody refers to one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An epitope tagged antibody refers to one wherein the antibody of the invention is fused to an epitope tag. The epitope tag polypeptide has enough residues to provide an epitope against which an antibody thereagainst can be made, yet is short enough such that it does not interfere with activity of the anti Hyphen antibody. The epitope tag preferably is sufficiently unique so that the antibody thereagainst does not substantially cross-react with other epitopes. Suitable tag polypeptides generally have at least 6 amino acid residues and usually between about 8-50 amino acid residues (preferably between about 9-30 residues). Examples include the flu HA tag polypeptide and its antibody 12CA5 (Field et al., Mol. Cell. Biol. 8: 2159-2165 (1988)); the c-myc tag and the 8F9, 3C7, 6E10, G4, B7 and 9E10 antibodies thereto (Evan et al., Mol. Cell. Biol. 5(12):3610-3616 (1985)); and the Herpes Simplex virus glycoprotein D (gD) tag and its antibody (Paborsky et al., Protein Engineering 3(6): 547-553 (1990)). In certain embodiments, the epitope tag is a salvage receptor binding epitope which is an epitope of the Fc region of an IgG molecule (e.g., IgG₁, IgG₂, IgG₃, or IgG₄) that is responsible for increasing the in vivo serum half-life of the IgG molecule.

Humanized antibodies as described herein include antibodies that have variable region frameworks derived from a human acceptor antibody molecule, hypervariable or CDR sequences from a donor murine antibody, and constant regions, if present, derived from human sequences.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody A Laboratory Manual, CSH Laboratories). The term antibody encompasses herein polyclonal, monoclonal antibodies and antibody variants such as single-chain antibodies, humanized antibodies, chimeric antibodies and immunologically active fragments of antibodies (e.g. Fab and Fab′ fragments) which inhibit or neutralize their respective interaction domains in Hyphen and/or are specific thereto.

Polyclonal antibodies are preferably raised in animals by multiple subcutaneous (sc), intravenous (iv) or intraperitoneal (ip) injections of the relevant antigen with or without an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals may be immunized against the antigen, immunogenic conjugates, or derivatives by combining the antigen or conjugate (e.g., 100 μg for rabbits or 5 μg for mice) with 3 volumes of Freund's complete adjuvant and injecting the solution intradermally at multiple sites. One month later the animals are boosted with the antigen or conjugate (e.g., with ⅕ to 1/10 of the original amount used to immunize) in Freund's complete adjuvant by subcutaneous injection at multiple sites. Seven to 14 days later the animals are bled and the serum is assayed for antibody titer. Animals are boosted until the titer plateaus. Preferably, for conjugate immunizations, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies may be made using the hybridoma method first described by Kohler et al., Nature, 256: 495 (1975), or may be made by recombinant DNA methods (e.g., U.S. Pat. No. 6,204,023). Monoclonal antibodies may also be made using the techniques described in U.S. Pat. Nos. 6,025,155 and 6,077,677 as well as U.S. Patent Application Publication Nos. 2002/0160970 and 2003/0083293 (see also, e.g., Lindenbaum, et al., Nucleic Acids Research 32 (21):0177 (2004)).

In the hybridoma method, a mouse or other appropriate host animal, such as a rat, hamster or monkey, is immunized (e.g., as hereinabove described) to elicit lymphocytes that produce or are capable of producing antibodies that will specifically bind to the antigen used for immunization. Alternatively, lymphocytes may be immunized in vitro. Lymphocytes then are fused with myeloma cells using a suitable fusing agent, such as polyethylene glycol, to form a hybridoma cell (see, e.g., Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOP-21 and M.C.-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (e.g., Kozbor, J. Immunol., 133: 3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can be determined, for example, by the Scatchard analysis of Munson et al., Anal. Biochem., 107: 220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures including, for example, protein A chromatography, hydrophobic interaction chromatography, hydroxylapatite chromatography, gel electrophoresis, dialysis, and/or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells, including those that do not otherwise produce immunoglobulin protein, to obtain the synthesis of monoclonal antibodies in the recombinant host cells.

There are a number of methods used to make antibodies human or human-like (e.g., “humanization”). Approaches to humanize antibodies have varied over the years. One approach was to generate murine variable regions fused to human constant regions, so-called murine-human Fc chimeras (see, e.g., Morrison et al, Proc. Natl. Acad. Sci. USA 81:6851-6855 (1984); U.S. Pat. No. 5,807,715). Another approach exploited the fact that CDRs could be readily identified based on their hypervariable nature (Kabat et al, J. Biol. Chem. 252:6609-6616 (1977)), Kabat, Adv. Protein Chem. 32:1-75 (1978)) and canonical structure (Chothia and Lesk, J. Mol. Biol. 196(4):901-17 (1987); Lazakani et al., J. Mol. Biol. 272:929 (1997) and humanized by grafting just the non-human CDR regions (referred to as donor CDRs) onto a human framework (referred to as acceptor frameworks) as shown, for example by Jones et al., Nature 321(6069):522-5 (1986); (see, e.g., U.S. Pat. No. 5,225,539; U.S. Pat. No. 6,548,640). The six CDR loops are presented in a cluster, and based on crystallographic analysis, critical framework residues within the so-called “Vernier” zone flanking the CDRs or in the heavy-light chain interface can be readily identified (see, e.g., Chothia and Lesk, J. Mol. Biol. 196(4):901-17 (1987); Chothia et al., J. Mol. Biol. 186(3):651-63 (1985); Chothia et al., Nature 342(6252):877-83 (1989)). These residues can be back-mutated to the murine residue to restore the correct relative orientation of the six CDRs (see, e.g., Verhoyen et al., Science 239(4847):1534-6 (1988); Reichman et al., Nature 332(6162):323-7 (1988); Tempest et al., Biotechnology (NY) 9(3):266-71 (1991)). Since variable regions can be classified in families that bear relatively high homology between mouse and human (reviewed in e.g., Pascual and Capra Adv. Immunol. 49:1-74 (1991)), these early studies also indicated that the potential for loss in affinity could be minimized in the grafted antibody by selecting the human germline sequence with the highest homology to the murine antibody of interest for use as the human acceptor molecule (see, e.g., U.S. Pat. No. 5,225,539; Verhoyen et al., Science 239(4847):1534-6 (1988)).

Family homologies and structural relationships between frameworks that impact correct presentation of a given type of CDR canonical structure have been reported (see, e.g., Al-Lazakani et al., J. Mol. Biol. 273(4):927-48 (1997) and references therein). Preferably, a best fit human or germline sequence is chosen. Available databases of antibody germline sequences may be used to determine the family subtype of a given murine heavy and light chain and to identify best fit sequences useful as human acceptor frameworks within that human subfamily. Both the linear amino acid homology of the donor and acceptor frameworks as well as the CDR canonical structure are preferably taken into account.

Another type of variant is an amino acid substitution variant. These variants have at least one amino acid residue in a humanized anti-Hyphen antibody molecule removed and a different residue inserted in its place. The sites of greatest interest for substitutional mutagenesis include the hypervariable loops, but framework alterations are also contemplated. Hypervariable region residues or framework residues involved in antigen binding are generally substituted in a relatively conservative manner. Such conservative substitutions are shown below under the heading of “preferred substitutions”. If such substitutions result in a change in biological activity, then more substantial changes, denominated “exemplary substitutions” or as further described below in reference to amino acid classes, are introduced and the products screened.

| Original | Exemplary | Preferred |
|---|---|---|
| Residue | Substitutions | Substitutions |
| Ala (A) | val; leu; ile | Val |
| Arg (R) | lys; gln; asn | Lys |
| Asn (N) | gln; his; lys; arg | Gln |
| Asp (D) | glu | Glu |
| Cys (C) | ser | Ser |
| Gln (Q) | asn | Asn |
| Glu (E) | asp | Asp |
| Gly (G) | pro; ala | Ala |
| His (H) | asn; gln; lys; arg | Arg |
| Ile (I) | leu; val; met; ala; | Leu |
| | phe; norleucine | |
| Leu (L) | norleucine; ile; val; | Ile |
| | met; ala; phe | |
| Lys (K) | arg; gln; asn | Arg |
| Met (M) | leu; phe; ile | leu |
| Phe (F) | leu; val; ile; ala; tyr | Leu |
| Pro (P) | ala | Ala |
| Ser (S) | thr | Thr |
| Thr (T) | ser | Ser |
| Trp (W) | tyr; phe | Tyr |
| Tyr (Y) | trp; phe; thr; ser | Phe |
| Val (V) | ile; leu; met; phe; | Leu |
| | ala; norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gin, his, lys, arg; (5) residues that influence chain orientation: gly, pro; and (6) aromatic: trp, tyr, phe.

Alternatively, amino acids are categorized as follows: small nonpolar (i.e. C, P, A and T), small polar (i.e. S, G, D and N), large polar (i.e. E, Q, K and R), intermediate polarity (i.e. Y, H and W), and large nonpolar (i.e. F, M, L, I and V).

Non-conservative substitutions will entail exchanging a member of one of these classes for another class. Any cysteine residue not involved in maintaining the proper confirmation of a humanized anti-Hyphen antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

Another type of amino acid variant of the antibody alters the original glycosylation pattern of the antibody. By altering is meant deleting one or more carbohydrate moieties found in the antibody and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition or deletion of glycosylation sites to the antibody is conveniently accomplished by altering the amino acid sequence such that it contains or lacks one or more of the above-described tripeptide sequences (for N-linked glycosylation sites). The alteration may also be made by the addition of, substitution by, or deletion of, one or more serine or threonine residues to the sequence of the original antibody (for O-linked glycosylation sites). Nucleic acid molecules encoding amino acid sequence variants of humanized anti-Hyphen antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, or cassette mutagenesis of an earlier prepared variant or a non-variant version of humanized anti-Hyphen antibody.

Ordinarily, amino acid sequence variants of a humanized anti-Hyphen antibody will have an amino acid sequence having at least 75% amino acid sequence identity with the original humanized antibody amino acid sequences of either the heavy or the light chain (e.g., variable region sequences as in SEQ ID NO:21 or SEQ ID NO:19, respectively), more preferably at least 80%, more preferably at least 85%, more preferably at least 90%, and most preferably at least 95%, including for example, 80%, 81%, 82%, 83%, 84%, 85%, 86%, 87%, 88%, 89%, 90%, 91%, 92%, 93%, 94%, 95%, 96%, 97%, 98%, 99%, and 100%. Identity or homology with respect to this sequence is defined herein as the percentage of amino acid residues in the candidate sequence that are identical with the humanized anti-Hyphen residues, after aligning the sequences and introducing gaps, if necessary, to achieve the maximum percent sequence identity, and not considering any conservative substitutions (as described above) as part of the sequence identity. None of N-terminal, C-terminal, or internal extensions, deletions, or insertions into the antibody sequence shall be construed as affecting sequence identity or homology. Thus sequence identity can be determined by standard methods that are commonly used to compare the similarity in position of the amino acids of two polypeptides. Using a computer program such as BLAST™ or FASTA™, two polypeptides are aligned for optimal matching of their respective amino acids (either along the full length of one or both sequences, or along a pre-determined portion of one or both sequences). The programs provide a default opening penalty and a default gap penalty, and a scoring matrix such as PAM250 (a standard scoring matrix; see Dayhoff et al., in Atlas of Protein Sequence and Structure, vol. 5, supp. 3 (1978)) can be used in conjunction with the computer program. For example, the percent identity can the be calculated as: the total number of identical matches multiplied by 100 and then divided by the sum of the length of the longer sequence within the matched span and the number of gaps introduced into the longer sequences in order to align the two sequences.

Antibodies having the characteristics identified herein as being desirable in a humanized anti-Hyphen antibody are screened for by methods as described herein. Cross-blocking assays can be performed and are described, for example, in Antibodies, A Laboratory Manual, Cold Spring Harbor Laboratory, Ed Harlow and David Lane (1988). In addition, or alternatively, epitope mapping, for example, as described in Champe et al., J. Biol. Chem. 270:1388-1394 (1995), can be performed to determine whether the antibody binds an epitope of interest.

Immobilized Hyphen can similarly be used to determine relative binding potencies by measuring Kᵢ values in competition assays. The inhibition curves are fitted with the “one site competition” model using Prism software (GraphPad, Inc. CA) to obtain IC₅₀ values and to calculate the Kᵢ using the equation of Cheng and Prusoff (Biochem, Pharmacol. 22(23):3099-108 (1973)).

In certain embodiments, the humanized anti-Hyphen antibody is an antibody fragment. Various techniques have been developed for the production of antibody fragments. Traditionally, these fragments were derived via proteolytic digestion of intact antibodies (see, e.g., Morimoto et al., Journal of Biochemical and Biophysical Methods 24: 107-117 (1992) and Brennan et al., Science 229: 81 (1985)). However, these fragments can be produced directly by recombinant host cells, such as bacteria (see, e.g., Better et al., Science 240(4855):1041-1043 (1988); U.S. Pat. No. 6,204,023. For example, Fab′-SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host cell culture. Other techniques for the production of antibody fragments will be apparent to the skilled practitioner.

As used herein, the terms “biological sample” refer to any sample from an animal that may be used to detect Hyphen proteins including antibodies or fragments thereof or Hyphen nucleic acids of the present invention. Without being so limited, these terms refer to blood, or a blood fraction such as serum or plasma, urine, saliva, tissue culture cell lines, sample containing in vitro-produced proteins, tissue including tumor tissue.

In the context of the present invention, an “isolated” or “substantially purified” DNA molecule or an “isolated” or “substantially purified” polypeptide is a DNA molecule or polypeptide that, by the hand of man, exists apart from its native environment and is therefore not a product of nature. An isolated DNA molecule or polypeptide may exist in a purified form or may exist in a non-native environment such as, for example, a transgenic host cell. An isolated or purified DNA or polypeptide may be synthesized chemically, may be produced using recombinant DNA techniques and then isolated or purified or may be isolated or purified from its natural host. An “isolated” or “substantially purified” nucleic acid molecule or protein, or biologically active portion thereof, is substantially free of other cellular material, or culture medium when produced by recombinant techniques and, in some circumstances, further purified, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein of interest chemicals.

The present invention also relates to a kit for detecting in or purifying from a biological sample a Hyphen protein, nucleic acid or a fragment thereof (Hyp product), and instructions to detect or purify the Hyp product in or from the sample. In addition, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample (DNA protein or cells), a container which contains the primers used in the assay, containers which contain enzymes, containers which contain wash reagents, and containers which contain the reagents used to detect the extension products.

### Assays

Based on the features described in the invention, it can be expected that methods to assay HYP products will be useful as detection and diagnostic reagents i.e. to detect, diagnose or monitor diseases such as cancer. Levels of HYP expression of the HYP gene may correlate with the occurrence of a cancer. Various immunological methods to assay soluble extracellular proteins are well known in the prior art (e.g. radioimmunoassays, ELISA, ‘sandwich’ ELISA). Such methods generally uses monoclonal or polyclonal antibodies specific to the protein of interest. In the case of the present invention, these antibodies could be raised against antigenic peptides and purified as described herein. An enzyme-linked immunoabsorbent assay can be used to quantify HYP products found in a sample. Other immunological methods to assay HYP products in biological tissues or fluids may be developed by a person skilled in the art. Various hybridization-based methods to assay for specific nucleic acids are well known in the prior art.

Proteins, peptides, or antibodies of the present invention can also be used for detecting and diagnosing cancer. For example, diagnosis can be accomplished by combining blood obtained from an individual to be tested with antibodies that specifically bind to HYP and determining the extent to which antibody is bound to the sample.

As used herein, the term “biological activity of Hyphen” includes without being so limited, Hyphen transcription, translation, post-translational modifications, degradation, subcellular localization, MEK phosphorylation, RAF activation, CNK/KSR binding, homotypic oligomerization, heterotypic oligomerization with CNK, Hyphen heterodimerization with CNK, KSR interaction, Hyphen-dependent signal transduction regulating, but not limited to, cell proliferation, cell differentiation and cell survival.

In accordance with one aspect of the present invention, there is thus provided a purified polypeptide comprising a SAM domain, the sequence of said SAM domain being as set forth in SEQ ID NO: 33. In a specific embodiment, the sequence of said SAM domain being as set forth in SEQ ID NO: 34. In an other specific embodiment, the sequence of said SAM domain being as set forth in SEQ ID NO: 35. In an other specific embodiment, the sequence of said SAM domain being as set forth in SEQ ID NO: 36. In an other specific embodiment, the sequence of said SAM domain being as set forth in SEQ ID NO: 37. In an other specific embodiment, the sequence of said SAM domain being as set forth in SEQ ID NO: 17. In an other specific embodiment, the sequence of said SAM domain being as set forth in SEQ ID NO: 18. In an other specific embodiment, the polypeptide is as set forth in SEQ ID NO: 1. In an other specific embodiment, the polypeptide is as set forth in SEQ ID NO: 2.

In accordance with an other aspect of the present invention, there is provided a purified antibody that specifically binds to a polypeptide of the present invention.

In accordance with an other aspect of the present invention, there is provided a method of determining whether a biological sample contains a polypeptide of the present invention, comprising contacting the sample with a purified ligand that specifically binds to the polypeptide, and determining whether the ligand specifically binds to the sample, the binding being an indication that the sample contains the polypeptide. In a specific embodiment, the ligand is a purified antibody that specifically binds to the polypeptide.

In accordance with an other aspect of the present invention, there is provided a method of purifying a polypeptide of the present invention from a biological sample containing the polypeptide, said method comprising: contacting the biological sample with a purified ligand that specifically binds to the polypeptide, the ligand being bound to a solid support, to produce a ligand-polypeptide complex, separating the complex from the remainder of the sample, and releasing the polypeptide from the ligand thereby obtaining the purified polypeptide. In a specific embodiment, the ligand is a purified antibody that specifically binds to the polypeptide.

In accordance with an other aspect of the present invention, there is provided a kit comprising a purified ligand that specifically binds to a polypeptide of the present invention, and instructions to use the ligand for detecting the polypeptide in a biological sample. In a specific embodiment, the ligand is a purified antibody that specifically binds to the polypeptide.

In accordance with an other aspect of the present invention, there is provided a kit comprising a purified ligand that specifically binds to a polypeptide of the present invention, and instructions to use the ligand for purifying the polypeptide from a biological sample. In a specific embodiment, the ligand is a purified antibody that specifically binds to the polypeptide.

In accordance with an other aspect of the present invention, there is provided an isolated polynucleotide encoding a polypeptide of the present invention.

In accordance with an other aspect of the present invention, there is provided a probe specifically hybridizable to a polynucleotide of the present invention.

In accordance with an other aspect of the present invention, there is provided a method of modulating RAS-mediated MAPK activation comprising modulating the biological activity of a polypeptide of the present invention. In a specific embodiment, said modulating is increasing the biological activity of the polypeptide.

In an other specific embodiment, said modulating is inhibiting the biological activity of the polypeptide. In an other specific embodiment, said step of inhibiting the biological activity of said polypeptide is conducted in vitro. In an other specific embodiment, said step of inhibiting the biological activity of said polypeptide is conducted in vivo. In an other specific embodiment, said step of inhibiting the biological activity of said polypeptide comprises administering a short interfering RNA molecule (siRNA) that decreases expression of said polypeptide. In an other specific embodiment, said step of inhibiting the biological activity of said polypeptide comprises administering a purified antibody that specifically binds to said polypeptide.

In accordance with an other aspect of the present invention, there is provided a short interfering RNA molecule (siRNA) that decreases the expression of a polypeptide by RNA interference, said polypeptide comprising a SAM domain, the sequence of said SAM domain being as set forth in SEQ ID NO: 37, comprising a sense region and an antisense region, wherein said antisense region comprises a sequence complementary to an RNA sequence encoding said polypeptide and the sense region comprises a sequence complementary to the antisense of said RNA sequence encoding said polypeptide. In a specific embodiment, said siRNA molecule is assembled from two nucleic acid fragments, wherein one fragment comprises the sense region and the second fragment comprises the antisense region of said siRNA molecule. said sense region and said antisense region are covalently connected via a linker molecule. In an other specific embodiment, said linker molecule is a polynucleotide linker molecule. In an other specific embodiment, said sense region comprises a 3′-terminal overhang of 1 to 5 nucleotides in length and said antisense region comprises a 3′-terminal overhang of 1 to 5 nucleotides in length. In an other specific embodiment, said sense and antisense regions comprise at least one nucleotide that is chemically modified in at least one of sugar, base, or backbone moiety. In an other specific embodiment, the siRNA molecule comprises a double stranded region of about 10 to 28 nucleotides in length. In an other specific embodiment, said siRNA molecule is linked to at least one receptor binding ligand. In an other specific embodiment, said receptor binding ligand is attached to the 5′-end, the 3′ end or both ends of the sense or antisense region of the siRNA molecule.

In accordance with an other aspect of the present invention, there is provided a method to identify a modulator of the polypeptide defined in claim 5, comprising: contacting a candidate compound with cells expressing said polypeptide; and assessing said cells for an alteration in a biological activity of said polypeptide, wherein a modulator of said polypeptide is identified when said biological activity is altered in the presence of the candidate compound as compared to in the absence thereof. In a specific embodiment, said modulator is an inhibitor and wherein an inhibitor of said polypeptide is identified when said biological activity is decreased in the presence of a candidate compound as compared to in the absence thereof. In an other specific embodiment, said biological activity is MEK activation.

In accordance with an other aspect of the present invention, there is provided a method of inhibiting Ras-dependent CNK RAF activation comprising inhibiting KSR activity.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows that the N-terminal portion of CNK augments KSR-induced MEK phosphorylation by RAF. (A) S2 cells were transfected using the indicated plasmid combinations. RAF activity was assessed by determining phospho-MEK levels in cell lysates by immunoblotting (α-pMEK). Protein levels were verified using the antibodies indicated to the right. Results presented here and thereafter are representative of at least three similar experiments. RAF and KSR kinase-mutant (KM) variants have a K455S and a K705M change, respectively. As previously reported (Douziech et al., 2003), co-expression of full-length CNK along with RAS^{V12} is inhibitory. This is caused by the RIR and is naturally alleviated by RTK-induced Src42 binding to the Y1163 region located C-terminal to the RIR (Laberge et al., 2005. EMBO J. 24: 487-98). To facilitate the characterization of the positive effect of the SAM and CRIC domains with respect to RAS-dependent RAF activation, NT-CNK constructs were used instead of full-length CNK, thereby bypassing the requirement in additional RTK signals. The SAM^{mut} NT-CNK variant has a L71K mutation, while the CRIC^{mut} variant has a three amino acid deletion (Del162S-H163-R164) that is similar to a mutation found in a cnk loss-of-function allele (Therrien et al., 1998). The difference between the effects produced by the mutations within the SAM domain and the CRIC region may be due to the fact that the CRIC mutation is hypomorphic while the L71K mutation obliterates normal SAM domain function. Alternatively, the involvement of the CRIC region may not be as decisive as the SAM domain. (B) Schematic of the main Drosophila RAF, KSR and CNK constructs used in this study. Full-length RAF contains a Ras-binding domain (RBD), a cysteine-rich domain (CRD) and a kinase domain (black box). The relative position of the S346 (S259-like), K455 (critical lysine in sub-domain II) and T571-T574 (phospho-accepting sites in the activation loop) residues is also shown. Full-length KSR comprises the so-called conserved area 1 (CA1; Therrien et al., 1995), a CRD and a putative kinase domain (black box). The conserved lysine residue of sub-domain II (K705) and the C922 residue that is critical for MEK binding (Roy et al., 2002) are also represented. Schematic of the N-terminal (1-655) and C-terminal (656-1003) KSR constructs are indicated as thick lines. Full-length CNK includes a SAM domain, a conserved region in CNK (CRIC), a PDZ domain, a proline-rich strech (Pro), a PH domain and a RAF-inhibitory Region (RIR) that includes two jointly required elements: a RAF-interacting Motif (RIM) and an Inhibitory Sequence (IS). NT-CNK (position 2-384), NT⁵⁴⁹-CNK (position 2-549), and CT-CNK (position 382-1557) constructs are depicted as thick lines;

FIG. 2 shows the specificity of RAF and KSR 3′UTR dsRNAs (i.e. RNAi). (A) Plain S2 cells were incubated or not (−) with the indicated 3′UTR dsRNAs for 4 days. Endogenous RAF or KSR was then monitored either from the cell lysates (RAF) or following immunoprecipitation (KSR) by immunoblot analysis. (B) A pyo-tagged RAF construct (50 ng) lacking the natural RAF 3′UTR sequences (D3′UTR) was transfected in S2 cells either alone (−) or with dsRNAs (500 ng) encompassing RAF open reading frame (ORF) or RAF 3′UTR sequences. The effect on pyoRAF expression was then determined by immunoblots using cell lysates. (C) As for pyoRAF, the expression in S2 cells of a V5-tagged KSR construct (30 ng) lacking its natural 3′UTR sequences, is unaffected by the presence KSR 3′UTR dsRNAs (500 ng);

FIG. 3 shows that CNK and KSR function upstream of the RAF activation loop phosphorylation event. S2 cells were transfected using the indicated plasmid combinations. (A-C) In each condition, dsRNA (500 ng) targeting the 3′UTR of endogenous RAF was also transfected. The RBD^{mut}, CRD^{mut}, AL^{ED} and AL^{AA} amino acid changes correspond to R174L, C249S-C252S, T571 E-T574D and T571A-T574A, respectively. (D) S2 cells were plated±RAF 3′UTR dsRNA (10 μg/ml) in combination with the other indicated dsRNAs (RNAi; 10 μg/ml each) and cultured for 24 h prior transfection (500 ng of the same dsRNAs was also added to the transfection mixture). RAF activity was evaluated by determining phospho-MAPK levels. GFP dsRNA is used as a negative control for RNAi. Addition of NT-CNK further reduced the mobility of RAF (compare lanes 3 and 4, α-PYO panel in A). This event is most likely due to phosphorylation as the mobility shifts could be eliminated by in vitro phosphatase treatment (data not shown). Although the phosphorylation sites and their functional relevance are unknown, there is a tight correlation between the positive effects of NT-CNK and RAF mobility shift, which was used as a second readout of NT-CNK effect on RAF;

FIG. 4 (A) RAF^{RBDmut} responds to RAS^{V12} activity in the presence of NT-CNK. S2 cells were co-transfected with RAF 3′UTR dsRNA (500 ng) and the indicated combinations of mycMEK^{DA} (70 ng), pyoRAF^{RBDmut} (80 ng), V5KSR (12 ng), haRAS^{V12} (30 ng) and NT-CNK (35 ng). Cell lysates were prepared 36 h post-induction of expression and pMEK levels were determined by immunoblots. (B) NT-CNK does not augment RAF^{ED} activity. S2 cells were co-transfected with RAF 3′UTR dsRNA (500 ng) and the indicated combinations of plasmids essentially as in (A), but with either wild-type pyoRAF (30 ng) (lanes 1 and 2) or the indicated amounts of pyoRAF-AL^{ED}. Cell lysates were also prepared 36 h post-induction of expression. Short and long exposures are shown for RAF level;

FIG. 5 shows that CNK activity is KSR-dependent. (A-B) S2 cells were transfected using the indicated plasmid combinations. dsRNA (500 ng) targeting the 3′UTR of KSR was also included in each condition. The CA1^{mut} amino acid change corresponds to L50S-R51G;

FIG. 6 shows that two inactive KSR mutants retain their ability to associate with RAF and MEK. S2 cells were transfected using the indicated plasmid combinations. (A-B) Cell lysates were immunoprecipitated using an α-V5 antibody to determine the amounts of MEK or RAF associated to KSR. Lysates were also directly probed to monitor protein levels. (C) RAF immunoprecipitation (α-PYO) was used to verify the capacity of KSR^{A696V-A703}T, compared to wild-type KSR, to promote the formation of a RAF/MEK complex;

FIG. 7 shows that CNK mediates the formation of a KSR/RAF complex. (A-C) S2 cells were transfected using the indicated plasmid combinations and cell lysates were either directly probed or immunoprecipitated as indicated. The star in (A) denotes the position of a non-specific protein revealed by the α-Flag antibody. (D) S2 cells were plated with the indicated dsRNAs (10 μg/ml). Four days later, cells were lysed and protein extracts were immunoprecipitated using a α-KSR monoclonal antibody. Two 100 mm dishes were pooled for each condition except for lane 7 where five dishes had to be pooled to obtain equal KSR protein levels as depletion of endogenous MEK greatly destabilized KSR (not shown). (E) S2 cells were incubated±dsRNA (RNAi; 10 μg/ml) directed at CNK 3′UTR sequences. Cells were transfected 24 h later using the indicated combinations of plasmids (+500 ng of CNK 3′UTR dsRNA) and processed as indicated;

FIG. 8 shows that the kinase domain of KSR interacts with endogenous CNK. (A) S2 cells were transfected with V5-KSR (100 ng), V5-KSR¹⁻⁶⁵⁵ (100 ng) or V5-KSR⁶⁵⁶⁻¹⁰⁰³ (50 ng). Cell lysates were prepared 36 h post-induction and immunoprecipitated using the anti-V5 antibody. Endogenous CNK associated to the KSR variants was then monitored by immunoblots using an anti-CNK antibody. (B) MEK overexpression restores the association between endogenous CNK (or NT⁵⁴⁹-CNK) and KSR variants. S2 cells were co-transfected with KSR 3′UTR dsRNA (500 ng) and the indicated combinations of V5-KSR (120 ng), V5-KSR^{DADA} (600 ng), V5-KSR^{C922Y} (450 ng), NT⁵⁴⁹-CNK (100 ng), with or without mycMEK^{DA} (400 ng). Cell lysates were prepared 36 h post-induction and either directly used to monitor protein levels or immunoprecipitated with anti-V5 to determine the amounts of both endogenous CNK and NT⁵⁴⁹-CNK associated with the KSR variants;

FIG. 9 shows that KSR recruitment to CNK depends on Hyphen (Hyp), a novel SAM domain-containing protein. (A) Depletion of endogenous HYP by RNAi reduces MAPK activation (pMAPK levels) induced by RAS^{V12}, but not by RAF-AL^{ED}. (B) HYP is required for MEK phosphorylation stimulated by NT-CNK. R—R—K-M denotes the co-transfected RAS^{V12}, RAF, KSR and MEK^{DA} plasmids. S2 cells in lanes 3 to 5 were plated with either dsGFP or dsHYP 3′UTR RNAs (6 μg/ml) for 24 h prior to transfection (500 ng of the same dsRNAs was included in the transfection mixture). (C) HYP is required for the CNK/KSR association. RNAi treatment was conducted as in (B). (D-E) HYP associates with CNK independently of KSR and requires the integrity of the SAM domain of CNK. A PYO-tagged HYP (A3′UTR) variant was used for these experiments as the AU5-tagged variant did not immunoprecipitate quantitatively. PYO—HYP also fully rescued MEK activation and CNK/KSR interaction following depletion of endogenous HYP (not shown). (E) Endogenous HYP mediates KSR/CNK complex formation in vivo. The experiment was conducted as in 5D;

FIG. 10 shows that Hyphen is an evolutionarily conserved protein (A) Dendrogram showing the phylogeny relationship among metazoan proteins closely related to Drosophila HYP. Database analysis uncovered a single gene in insects (Drosophila CG30476; A. gam. [EAA00520.2]; A. mel. [Ensembl access #: ENSAPMP00000022190]) and nematode (C. ele. [M01G5.6]), while multiple homologues seem to exist in higher metazoans such as in mammals (Mmus12: NP_{—}796199 and Mmus10: NP_{—}766264); birds (Ggal1: XP_{—}418395.1; and Ggal2: XP_{—}425711.1) and fish (Tnig1: CAG11091.1; Tnig2: CAG11266.1; Tnig3: CAG03919.1). (B) Amino acid comparison of the insects (Dmel (SEQ ID NO: 15); Agam (SEQ ID NO: 35); Amel (SEQ ID NO: 36)), nematode (Cele (SEQ ID NO: 16)) and mouse HYP homologues (Mmus12 (SEQ ID NO: 9) and Mmus10 (SEQ ID NO: 10)). (C) Pyo-tagged HYP constructs (100 ng) containing or lacking (D3′UTR) HYP 3′UTR sequences were transfected in S2 cells either alone (−) or with dsRNAs (500 ng) encompassing HYP open reading frame (ORF) or HYP 3′UTR sequences. The effect of the dsRNAs on pyoHYP expression was then monitored by immunoblots following immunoprecipitation (IP) of pyoHYP;

FIG. 11 schematically shows a model summarizing the scaffolding and the RTK-dependent activating property of the KSR/HYP/CNK complex with respect to RAF and MEK. For simplicity, the model (detailed in the text) does not include the presumed effects of 14-3-3 protein-binding on RAF and KSR conformation and localization. Dotted arrows represent possible entry points for RAS activity that is integrated by the complex independently of the RAF RBD. The arrow with a question mark between KSR and RAF kinase domains illustrates the putative RAS-dependent RAF-activating effect of KSR in association with CNK/HYP;

FIG. 12 presents Hyphen homologues from various species, namely human Hsap1 (SEQ ID NO: 1) and Hsap2 (SEQ ID NO: 2); Chimpanzee Ptro1 (SEQ ID NO: 3) and Ptro2 (SEQ ID NO: 4); Dog Cfam1 (SEQ ID NO: 5) and Cfam2 (SEQ ID NO: 6); cow Btau1 (SEQ ID NO: 7) and Btau2 (SEQ ID NO: 8); mouse Mmus1 (SEQ ID NO: 9) and Mmus2 (SEQ ID NO: 10); rat Rnor1 (SEQ ID NO: 11) and Rnor2 (SEQ ID NO: 12); chicken Ggal1 (SEQ ID NO: 13) and Ggal2 (SEQ ID NO: 14); Drosophila Dmel (SEQ ID NO: 15); and nematode Cele (SEQ ID NO: 16) wherein Hsap: Homo sapiens (human); Ptro: Pan troglodytes (chimpanzee); Cfam: Canis familiaris (dog); Btau: Bos taurus (cow); Mmus: Mus musculus (mouse); Rnor: Ratus norvegicus (rat); Ggal: Gallus gallus (chicken); Dmel: Drosophila melanogaster (fruitfly); Cele: Caenorhabditis elegans (nematode). Note₁: Contrarily to Drosophila (Dmel) and Nematode (Cele), mammals and birds (Ggal) have two genes (identified 1 and 2) encoding Hyphen homologues. Note₂: Type 1 homologues are also designated under the name Samd10 in NCBI, while type 2 homologues correspond to Samd12;

FIG. 13 shows an alignment of the Hyp homologues presented in FIG. 12. Note: the SAM domain (highly conserved between homologues) starts at position 371 of the alignment (which corresponds to valine-115 in the human Hsap1 homologue) and ends at position 446 of the alignment (which corresponds to glutamate-185 in the human Hsap1 homologue); and SAM domains are thus identifiable in each of the homologues and given a sequence ID number as follows: Hsap1 (SEQ ID NO: 17) and Hsap2 (SEQ ID NO: 18); Chimpanzee Ptro1 (SEQ ID NO: 19) and Ptro2 (SEQ ID NO: 20); Dog Cfam1 (SEQ ID NO: 21) and Cfam2 (SEQ ID NO: 22); cow Btau1 (SEQ ID NO: 23) and Btau2 (SEQ ID NO: 24); mouse Mmus1 (SEQ ID NO: 26) and Mmus2 (SEQ ID NO: 26); rat Rnor1 (SEQ ID NO: 27) and Rnor2 (SEQ ID NO: 28); chicken Ggal1 (SEQ ID NO: 29) and Ggal2 (SEQ ID NO: 30); Drosophila Dmel (SEQ ID NO: 31); and nematode Cele (SEQ ID NO: 32). A consensus sequence derived from the sequences of the above-disclosed species is designated SEQ ID NO: 33 (xxxwxxxdvxxwxxxxxxxxxxxyxxxfxxhxxtgrxlxxxxxxxlxxxgxxxxxxrxxxxxxxxxxxxxx) while a consensus sequence derived from the sequences of the two human Hyp homologues is identified SEQ ID NO: 34 (vxlwxqqdvckwlkkhcpxxyxxyxexfxqhxitgrallrlxxxklxrmgxaqexxrqxxlqqvlxlxvre), a consensus sequence derived from the sequences of the above-disclosed species (i.e. excluding the nematode) is identified SEQ ID NO: 35 (vxlwxxxdvxkwxxxhcxxxxxxyxxxfxxhxitgrallrlxxxxlxrmgxxxxxxrxxxxxxxxxxxxxx), the consensus sequence derived from the sequences of the above-disclosed animal species (i.e. excluding the nematode and fly) is identified SEQ ID NO: 36 (vxlwxqqdvckwlkkhcpxxyxxyxexfxxhxitgrallrlxxxklxrmgxxxexxrqxxlqqvlxlxvre), while the consensus sequence derived from the sequences of the above-disclosed mammal species (i.e. excluding the nematode, fly and chicken) is identified SEQ ID NO: 37 (vxlwxqqdvckwlkkhcpxxyxxyxexfxqhxitgrallrlxxxklxrmgxxqexxrqxxlqqvlxlxvre). These consensus sequences contain at each position a residue that is absent or selected from those found at that position in disclosed Hyphen homologues;

FIG. 14 presents a cladogram illustrating the phylogenetic relationship between Hyphen homologues described in FIG. 12;

FIG. 15 presents the results of a co-immunoprecipitation of mouse Hyphen homologues Samd10 and Samd12 with Drosophila CNK. Mouse Hyphen homologues associate with NT-CNK. When separated on SDS-PAGE, NT-CNK was resolved as two discrete bands due to phosphorylation events (not shown). The presence of Drosophila Hyphen (not shown) or the mouse isoforms increases the level of the slower migrating form of NT-CNK. Interestingly, while Samd10 appears to associate with both NT-CNK species, Samd12 seems to associate specifically with the slower migrating form; and

FIG. 16 presents the results of a functional assay demonstrating the ability of mouse Hyphen homologues Samd10 and Samd12 to complement the activity of Drosophila Hyphen in RAS-mediated MEK activation.

### DESCRIPTION OF ILLUSTRATIVE EMBODIMENTS siRNAs

The present invention further concerns the use of RNA interference (RNAi) to decrease HYP expression in target cells. “RNA interference” refers to the process of sequence specific suppression of gene expression mediated by small interfering RNA (siRNA) without generalized suppression of protein synthesis. While the invention is not limited to a particular mode of action, RNAi may involve degradation of messenger RNA (e.g., HYP mRNA) by an RNA induced silencing complex (RISC), preventing translation of the transcribed targeted mRNA. Alternatively, it may involve methylation of genomic DNA, which shuts down transcription of a targeted gene. The suppression of gene expression caused by RNAi may be transient or it may be more stable, even permanent.

RNA interference is triggered by the presence of short interfering RNAs of about 20-25 nucleotides in length which comprise about 19 base pair duplexes. These siRNAs can be of synthetic origin or they can be derived from a ribonuclease III activity (e.g., dicer ribonuclease) found in cells. The RNAi response also features an endonuclease complex containing siRNA, commonly referred to as an RNA-induced silencing complex (RISC), which mediates the cleavage of single stranded RNA having a sequence complementary to the antisense region of the siRNA duplex. Cleavage of the target RNA (e.g., HYP mRNA) takes place in the middle of the region complementary to the antisense strand of the siRNA duplex (Elbashir et al., 2001, Genes Dev., 15:188).

“Small interfering RNA” of the present invention refers to any nucleic acid molecule capable of mediating RNA interference “RNAi” or gene silencing (see for example, Bass, 2001, Nature, 411:428-429; Elbashir et al., 2001, Nature, 411:494-498; Kreutzer et al., International PCT publication No. WO 00/44895; Zernicka-Goetz et al., International PCT publication No. WO 01/36646; Fire, International PCT publication No. WO99/32619; Mello and Fire, International PCT publication No. WO01/29058; Deschamps-Depaillette, International PCT publication No. WO99/07409; Han et al., International PCT puplication No. WO 2004/011647; Tuschl et al., International PCT publication No. WO 02/44321; and Li et al., International PCT publication No. WO 00/44914). For example, siRNA of the present invention are double stranded RNA molecules from about ten to about 30 nucleotides long that are named for their ability to specifically interfere with protein expression. In one embodiment, siRNA of the present invention are 12-28 nucleotides long, more preferably 15-25 nucleotides long, even more preferably 19-23 nucleotides long and most preferably 21-23 nucleotides long. Therefore preferred siRNA of the present invention are 12, 13, 14, 15, 16, 17, 18, 19, 20, 21, 22, 23, 24, 25, 26, 27, 28 nucleotides in length. As used herein, siRNA molecules need not to be limited to those molecules containing only RNA, but further encompass chemically modified nucleotides and non-nucleotides. It is important to note that dsRNA used for targeting Hyphen in the Drosophila in examples herein are not siRNA but 400 to 800 bp dsRNA which can advantageously be used for RNAi in Drosophila.

The length of one strand designates the length of an siRNA molecule. For example, a siRNA that is described as a 23 ribonucleotides long (a 23 mer) could comprise two opposite strands of RNA that anneal together for 21 contiguous base pairing. The two remaining ribonucleotides on each strand would form what is called an “overhang”. In a particular embodiment, the siRNA of the present invention contains two strands of different lengths. In this case, the longer strand designates the length of the siRNA. For example, a dsRNA containing one strand that is 20 nucleotides long and a second strand that is 19 nucleotides long is considered a 20 mer.

siRNAs that comprise an overhang are desirable. The overhang may be at the 3′ or 5′ end. Preferably, the overhangs are at the 3′ end of an RNA strand. The length of an overhang may vary but preferably is about 1 to 5 nucleotides long. Generally, 21 nucleotides siRNA with two nucleotides 3′-overhang are the most active siRNAs.

siRNA of the present invention are designed to decrease HYP expression in a target cell by RNA interference. siRNA of the present invention comprise a sense region and an antisense region wherein the antisense region comprises a sequence complementary to a HYP mRNA sequence and the sense region comprises a sequence complementary to the antisense sequence of HYP mRNA. A siRNA molecule can be assembled from two nucleic acid fragments wherein one fragment comprises the sense region and the second fragment comprises the antisense region of siRNA molecule. The sense region and antisense region can also be covalently connected via a linker molecule. The linker molecule can be a polynucleotide linker or a non polynucleotide linker.

In one embodiment, the present invention features a siRNA molecule having RNAi activity against HYP RNA, wherein the siRNA molecule comprises a sequence complementary to any RNA having a HYP encoding sequence. A siRNA molecule of the present invention can comprise any contiguous HYP sequence (e.g. 19-23 contiguous nucleotides present in a HYP sequence). In the particular case where alternate splicing produces a family of transcripts that are distinguished by specific exons, the present invention can be used to inhibit gene expression of a particular gene family member through the targeting of the appropriate exon(s) (e.g., to specifically knock down the expression of a HYP particular transcript) or of the full length transcript.

siRNAs of the present invention comprise a ribonucleotide sequence that is at least 80% identical to a HYP ribonucleotide sequence. Preferably, the siRNA molecule is at least 90%, at least 95% (e.g., 95, 96, 97, 99, 99, 100%), at least 98% (e.g., 98, 99, 100%) or at least 99% (e.g., 99, 100%) identical to the ribonucleotide sequence of the target gene (e.g., HYP RNA). siRNA molecule with insertion, deletions, or single point mutations relative to the target may also be effective. Mutations that are not in the center of the siRNA molecule are more tolerated. Tools to assist siRNA design are well known in the art and readily available to the public. For example, a computer-based siRNA design tool is available on the Internet at www.dharmacon.com or are available on the web site of several companies that offer the synthesis of siRNA molecules.

In one embodiment, the siRNA molecules of the present invention are chemically modified to confer increased stability against nuclease degradation but retain the ability to bind to the target nucleic acid that is present in a cell. Modified siRNAs of the present invention comprise modified ribonucleotides, and are resistant to enzymatic degradation such as RNAse degradation, yet they retain their ability to reduce HYP expression in a target cell. The siRNA may be modified at any position of the molecule so long as the modified siRNA is still capable of binding to the target sequence and is more resistant to enzymatic degradation. Modifications in the siRNA may be in the nucleotide base (i.e., purine or pyrimidine), the ribose or phosphate.

More specifically, the siRNA may be modified in at least one purine, in at least one pyrimidine or a combination thereof. Generally, all purines (adenosine or guanine) or all pyrimidine (cytosine or uracyl) or a combination of all purines and all pyrimidines of the siRNA are modified. Ribonucleotides on either one or both strands of the siRNA may be modified.

Non-limiting examples of chemical modification that can be included in an siRNA molecule include phosphorothioate internucleotide linkages (see US 2003/0175950), 2′-O-methyl ribonucleotides, 2′-O-methyl modified ribonucleotides, 2′-deoxy-2′-fluoro ribonucleotides, 2′-deoxy-2′-fluoro modified pyrimidines nucleotides, 5-C-methyl nucleotides and deoxyabasic residue incorporation. The ribonucleotides containing pyrimidine bases can be modified at the 2′ position of the ribose residue. A preferable modification is the addition of a molecule from the halide chemical group such as fluorine. Other chemical moieties such as methyl, methoxymethyl and propyl may also be added as modifications (see International PCT publication No. WO2004/011647). These chemical modifications, when used in various siRNA constructs, are shown to preserve RNAi activity in cells while at the same time, dramatically increasing their stability in cells or serum. Chemical modifications of the siRNA of the present invention can also be used to improve the stability of the interaction with the target RNA sequence.

siRNAs of the present invention may also be modified by the attachment of at least one receptor binding ligand to the siRNA. Receptor binding ligand can be any ligand or molecule that directs the siRNA of the present invention to a specific target cell (e.g., NK cells, macrophage, dendritic cells). Such ligands are useful to direct delivery of siRNA to a target cell in a body system, organ or tissue of a subject such as NK cells. Receptor binding ligand may be attached to one or more siRNA ends, including any combination of 5′ or 3′ ends. The selection of an appropriate ligand for delivering siRNAs depends on the cells, tissues or organs that are targeted and is considered to be within the ordinary skill of the art. For example, to target a siRNA to hepatocytes, cholesterol may be attached at one or more ends, including 3′ and 5′ ends. Other conjugates such as other ligands for cellular receptors (e.g., peptides derived from naturally occurring protein ligands), protein localization sequences (e.g., ZIP code sequences), antibodies, nucleic acid aptamers, vitamins and other cofactors such as N-acetylgalactosamine and folate, polymers such as polyethyleneglycol (PEG), polyamines (e.g., spermine or spermidine) and phospholipids can be linked (directly or indirectly) to the siRNA molecule for improving its bioavailability.

siRNAs can be prepared in a number of ways well known in the art, such as by chemical synthesis, T7 polymerase transcription, or by treating long double stranded RNA (dsRNA) prepared by one of the two previous methods with Dicer enzyme. Dicer enzyme create mixed population of dsRNA from about 21 to 23 base pairs in length from double stranded RNA that is about 500 base pairs to about 1000 base pairs in size. Dicer can effectively cleave modified strands of dsRNA, such as 2′-fluoromodified dsRNA (see WO2004/011647).

In one embodiment, vectors are employed for producing siRNAs by recombinant techniques. Thus, for example, a DNA segment encoding a siRNA derived from a HYP sequence may be included in anyone of a variety of expression vectors for expressing any DNA sequence derived from a HYP sequence. Such vectors include synthetic DNA sequences (e.g., derivatives of SV40, bacterial plasmids, baculovirus, yeast plasmids, viral DNA such as vaccinia, fowl pox virus, adenovirus, lentivirus, retrovirus, adeno-associated virus, alphavirus etc), chromosomal, and non chromosomal vectors. Any vector may be used in accordance with the present invention as long as it is replicable and viable in the desired host. The DNA segment in the expression vector is operatively linked to an appropriate expression control sequence(s) (e.g., promoter) to direct siRNA synthesis. Preferably, but not exclusively (Pol II promoters could also be exploited. See Dickins et al., (2005) Nat. Genet. 37, 1289-1295), the promoters of the present invention are from the type III class of RNA polymerase III promoters (e.g., U6 and H1 promoters). The promoters of the present invention may also be inducible, in that the expression may be turned on or turned off (e.g., tetracycline-regulatable system employing the U6 promoter to control the production of siRNA targeted to HYP).

In a particular embodiment, the present invention utilizes a vector wherein a DNA segment encoding the sense strand of the RNA polynucleotide is operatebly linked to a first promoter and the antisense strand of the RNA polynucleotide is operably linked to a second promoter (i.e., each strand of the RNA polynucleotide is independently expressed).

In another embodiment, the DNA segment encoding both strands of the RNA polynucleotide are under the control of a single promoter. In a particular embodiment, the DNA segment encoding each strand are arranged on the vector with a loop region connecting the two DNA segments (e.g., sense and antisense sequences), where the transcription of the DNA segments and loop region creates one RNA transcript. When transcribed, the siRNA folds back on itself to form a short hairpin capable of inducing RNAi. The loop of the hairpin structure is preferably from about 4 to 6 nucleotides in length. The short hairpin is processed in cells by endoribonucleases which removes the loop thus forming a siRNA molecule. In this particular embodiment, siRNAs of the present invention comprising a hairpin or circular structures are about 35 to about 65 nucleotides in length (e.g., 35, 36, 37, 38, 49, 40, 41, 42, 43, 44, 45, 46, 47, 48, 49, 50, 51, 52, 53, 54, 55, 56, 57, 58, 59, 60, 61, 63, 64, 65 nucleotides in length), preferably between 40 and 64 nucleotides in length comprising for example about 18, 19, 20, 21, 22, or 23, 24, 25 base pairs.

In yet a further embodiment, the vector of the present invention comprises opposing promoters. For example, the vector may comprise two RNA polymerase III promoters on either side of the DNA segment (e.g., a specific HYP DNA segment) encoding the sense strand of the RNA polynucleotide and placed in opposing orientations, with or without a transcription terminator placed between the two opposing promoters.

Non-limiting examples of expression vectors used for siRNA expression are described in Lee et al., 2002, Nature Biotechnol., 19:505; Miyagishi and Taira, 2002, Nature Biotechnol., 19:497; Pau et al., 2002, Nature Biotechnol., 19:500 and Novina et al., 2002, Nature Medecine, July 8(7):681-686). PoIII

### Antisense RNAs

The present invention also features antisense nucleic acid molecules which can be used for example to decrease or abrogate the expression of HYP. An antisense nucleic acid molecule according to the present invention refers to a molecule capable of forming a stable duplex or triplex with a portion of its targeted nucleic acid sequence (DNA or RNA). The use of antisense nucleic acid molecules and the design and modification of such molecules is well known in the art as described for example in WO 96/32966, WO 96/11266, WO 94/15646, WO 93/08845, and U.S. Pat. No. 5,593,974. Antisense nucleic acid molecules according to the present invention can be derived from the nucleic acid sequences and modified in accordance to well known methods. For example, some antisense molecules can be designed to be more resistant to degradation to increase their affinity to their targeted sequence, to affect their transport to chosen cell types or cell compartments, and/or to enhance their lipid solubility by using nucleotide analogs and/or substituting chosen chemical fragments thereof, as commonly known in the art.

In one embodiment, antisense approach of the present invention involves the design of oligonucleotides (either DNA or RNA) that are complementary to HYP mRNA. The antisense oligonucleotides bind to HYP mRNA and prevent its translation. Absolute complementarity, although preferred, is not a definite prerequisite. One skilled in the art can identify a certain tolerable degree of mismatch by use of standard methods to determine the melting point of the hybridized antisense complex. In general, oligonucleotides that are complementary to the 5′untranslated region (up to the first AUG initiator codon) of HYP mRNA should work more efficiently at inhibiting translation and production of HYP protein. However, oligonucleotides that are targeted to a coding portion of the sequence may produce inactive truncated protein or diminish the efficiency of translation thereby lowering the overall expression of HYP protein in a cell. Antisense oligonucleotides targeted to the 3′ untranslated region of messages have also proven to be efficient in inhibiting translation of targeted mRNAs (Wagner, R. (1994), Nature, 372:333-335). The HYP antisense oligonucleotides of the present invention are less than 100 nucleotides in length, particularly, less than 50 nucleotides in length and more particularly less than 30 nucleotides in length. Generally, effective antisense oligonucleotides are at least 15 or more oligonucleotides in length.

The antisense oligonucleotides of the present invention can be DNA, RNA, Chimeric DNA-RNA analogue, and derivatives thereof (see Inoue et al. (1987), Nucl. Acids. Res. 15: 6131-6148; Inoue et al. (1987), FEBS lett. 215: 327-330; Gauthier at al. (1987), Nucl. Acids, Res. 15: 6625-6641.). As mentioned above, antisense oligonucleotides of the present invention may include modified bases or sugar moiety. Examples of modified bases include xanthine, hypoxanthine, 2-methyladenine, N6-isopentenyladenine, 2-methylguanine, 3-methylcytosine, 5-methylcytosine, N6-adenine, 7-methyguanine, 5-fluorouracil, 5-chlorouracil, 5-bromouracil, 5-iodouracyl, 5-carboxymethylaminomethyluracil, 5-methoxycarboxymethyluracil, queosine, 4-thiouracil and 2,6-diaminopurine. Examples of modified sugar moieties include hexose, xylulose, arabinose and 2-fluoroarabinose. The antisense oligonucleotides of the present invention may also include modified phosphate backbone such as methylphosphonate, phosphoramidate, phosphoramidothioate, phosphordiamidate and alkyl phosphotriesters. The synthesis of modified oligonucleotides can be done according to methods well known in the art.

Once an antisense oligonucleotide or siRNA is designed, its effectiveness can be appreciated by conducting in vitro studies that assess the ability of the antisense to inhibit gene expression (e.g., HYP protein expression). Such studies ultimately compare the level of HYP RNA or protein with the level of a control experiment (e.g., an oligonucleotide which is the same has that of antisense experiment but being a sense oligonucleotide or an oligonucleotide of the same size as the antisense oligonucleotide but that does not bind to a specific HYP sequence).

### Increase HYP Expression

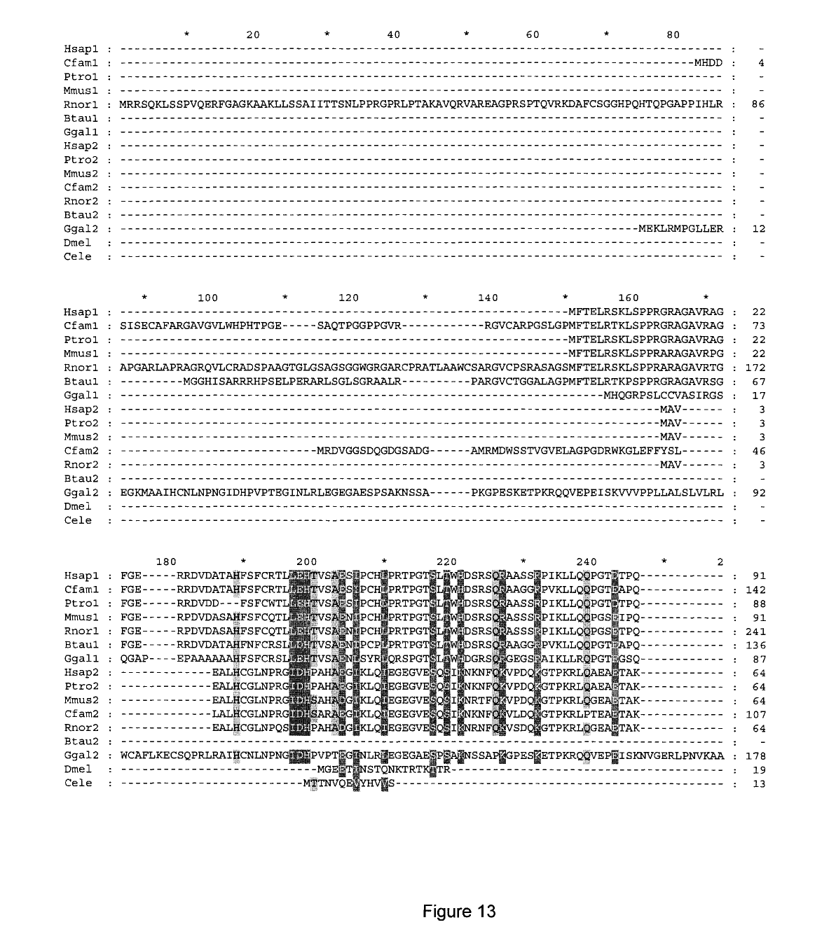

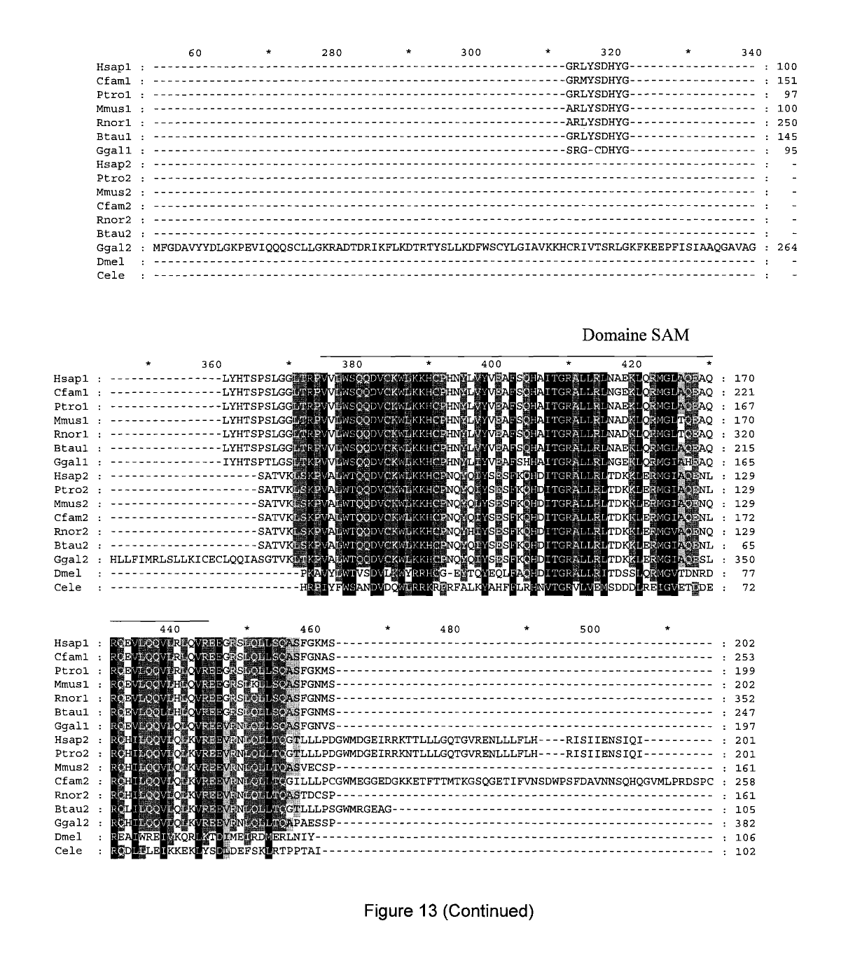

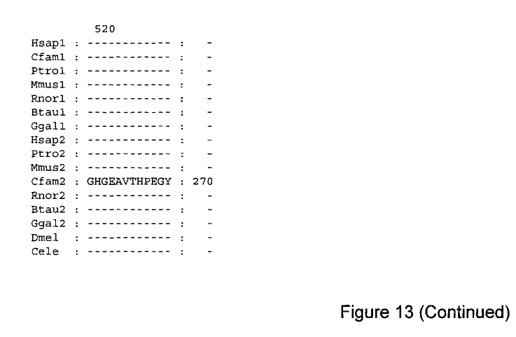

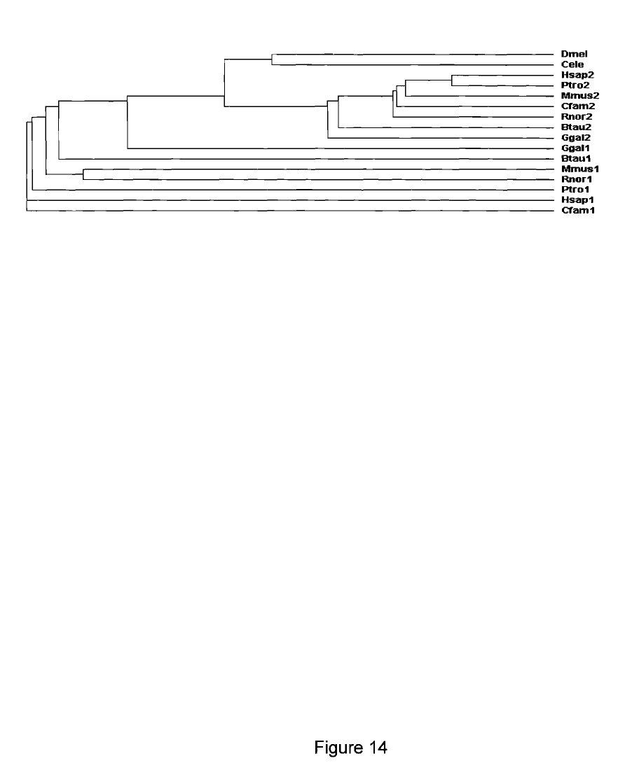

In particular conditions it might be useful to stimulate or increase the expression of HYP in cells. Thus, in one particular embodiment, the present invention features gene therapy methods to increase HYP expression in cells. The HYP sequences used in the gene therapy method of the present invention may be either a full-length HYP nucleic acid sequence (e.g. encoding a polypeptide of FIG. 13) or be limited to sequences encoding the biologically active domains of a HYP protein. Thus, any HYP sequence having at least one conserved biological activity of native HYP protein may be used in accordance with the present invention. The HYP sequence may be under the control of its natural promoter or under the control of other strong promoter allowing either general expression or cell-type or tissue specific expression.

### Gene Therapy Methods

In the gene therapy methods of the present invention an exogenous sequence (e.g., a HYP gene or cDNA sequence, a HYP siRNA or antisense nucleic acid) is introduced and expressed in an animal (preferably a human) to supplement, replace or inhibit a target gene (i.e., HYP gene), or to enable target cells to produce a protein having a prophylactic or therapeutic effect toward cancers or other diseases characterized by an unrestrained signalling through RAS/MAPK pathway.

Non virus-based and virus-based vectors (e.g., adenovirus- and lentivirus-based vectors) for insertion of exogenous nucleic acid sequences into eukaryotic cells are well known in the art and may be used in accordance with the present invention. Virus-based vectors (and their different variations) for use in gene therapy are well known in the art. In virus-based vectors, parts of a viral gene are replaced by the desired exogenous sequence so that a viral vector is produced. Viral vectors are very often designed to no longer be able to replicate due to DNA manipulations.

In one specific embodiment, lentivirus derived vectors are used to target an anti-HYP sequence (e.g., siRNA, antisense, nucleic acid encoding a partial or complete HYP protein) into specific target cells. These vectors have the advantage of infecting quiescent cells (for example see U.S. Pat. No. 6,656,706; Amado et al., 1999, Science 285: 674-676).

In addition to a HYP nucleic acid sequence, siRNA or antisense, the vectors of the present invention may contain a gene that acts as a marker by encoding a detectable product.

One way of performing gene therapy is to extract cells from a patient, infect the extracted cells with a viral vector and reintroduce the cells back into the patient. A selectable marker may or may not be included to provide a means for enriching the infected or transduced cells. Alternatively, vectors for gene therapy that are specially formulated to reach and enter target cells may be directly administered to a patient (e.g., intravenously, orally etc.).

The exogenous sequences (e.g. antisense RNA, siRNA, a HYP sequence, or HYP targeting vector for homologous recombination) may be delivered into cells that express HYP according to well known methods. Apart from infection with virus-based vectors, examples of methods to deliver nucleic acid into cells include DEAE dextran lipid formulations, liposome-mediated transfection, CaCl₂-mediated transfection, electroporation or using a gene gun. Synthetic cationic amphiphilic substances, such as dioleoyloxypropylmethylammonium bromide (DOTMA) in a mixture with dioleoylphosphatidylethanolamine (DOPE), or lipopolyamine (Behr, Bioconjugate Chem., 1994 5:382), have gained considerable importance in charged gene transfer. Due to an excess of cationic charge, the substance mixture complexes with negatively charged genes and binds to the anionic cell surface. Other methods include linking the exogenous oligonucleotide sequence (e.g., siRNA, antisense, HYP sequence encoding a HYP protein, HYP targeting vector for homologous recombination, etc) to peptides or antibodies that especially binds to receptors or antigens at the surface of a target cell. U.S. Pat. No. 6,358,524 describes target cell-specific non-viral vectors for inserting at least one gene into cells of an organism. The method describes the uses of non-viral carriers that are cationized to enable them to complex with the negatively charged DNA. Moreover, the method also includes the use of a ligand (e.g., antibody or fragment thereof that is specific in this particular case for a HYP protein) can specifically bind to the desired target cell in order to enter it.

To achieve high cellular concentration of the HYP antisense nucleic acid or siRNAs of the present invention an effective method utilizes a recombinant DNA construct in which the nucleic acid sequence is placed under a strong promoter and the entire construct is targeted into the cell. Such promoter may constitutively or inducibly produce the HYP sequence encoding HYP protein (or portion thereof), antisense RNA or siRNA of the present invention.

### Assays to Identify Modulators of HYP

In order to identify modulators of HYP biological activity, several screening assays aiming at reducing, abrogating or stimulating a functional activity of HYP in cells can be designed in accordance with the present invention.

One possible way is by screening libraries of candidate compounds for inhibitors of HYP biological activity. In particular, it includes screening for compounds that inhibit the interaction of HYP with SAM-related receptors (e.g. CNK). Inhibitors of other HYP functional activities may also be identified in accordance with the present invention. In addition, libraries of candidate compounds may also be screened for stimulators of HYP activity (e.g. compounds which increase the binding of HYP to CNK, or other HYP biological activity). Screening assays and compounds which directly or indirectly modulate (i.e. decrease or increase) HYP expression in cells are also encompassed by the present invention.

For example, combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection may be used in order to identify modulators of HYP biological activity. The biological library approach is limited to peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be found in the art, for example in: DeWitt et al. (1993) Proc. Natl. Acad. Sci. USA. 90:6909; Erb et al. (1994) Proc. Natl. Acad. Sci. USA 91:11422; Zuckermann et al. (1994), J. Med. Chem. 37:2678; Cho et al. (1993) Science 261:1303; Carrell et al. (1994) Angew. Chem., Int. Ed Engl. 33:2059; and ibid 2061; and in Gallop et al. (1994). Med. Chem. 37:1233. Libraries of compounds may be presented in solution (e.g. Houghten (1992) Biotechniques 13:412-421) or on beads (Lam (1991) Nature 354:82-84), chips (Fodor (1993) Nature 364:555-556), bacteria or spores (Ladner U.S. Pat. No. 5,223,409), plasmids (Cull et al., (1992) Proc Natl Acad Sci USA 89:1865-1869) or on phage (Scott and Smith (1990); Science 249:386-390). Examples of methods for the synthesis of molecular libraries can be found in the art, for example in: DeWitt et al. (1993) supra; Erb et al. (1994) supra; Zuckermann et al. (1994) supra; Cho et al. (1993) supra; Carrell et al. (1994) supra, or luciferase, and the enzymatic label detected by determination of conversion of an appropriate substrate to product. The choice of a particular combinatorial library depends on the specific HYP activity that needs to be modulated.

The SAM domain of HYP has been identified as one of the functional regions of HYP. Thus, to reduce HYP activity in cells, the present invention concerns the use of assays allowing rapid identification of small molecules that inhibit HYP binding activity to SAM related receptors. Similarly, screening assays that will permit rapid identification of small molecules that inhibit activities downstream of HYP/CNK binding are encompassed by the present invention.

All methods and assays of the present invention may be developed for low-throughput, high-throughput, or ultra-high throughput screening formats. Of course, methods and assays of the present invention are amenable to automation. Automation and low-throughput, high-throughput, or ultra-high throughput screening formats are possible for the screening of agents which modulates the level and/or activity of HYP.

Generally, high throughput screens for HYP modulators i.e. candidate or test compounds or agents (e.g., peptides, peptidomimetics, small molecules, antisense RNA, siRNA, Ribozyme, or other drugs) may be based on assays which measure a biological activity of HYP. The invention therefore provides a method (also referred to herein as a “screening assay”) for identifying modulators, which have an inhibitory effect on, for example, an HYP biological activity or expression thereof, or which bind to or interact with HYP proteins.

The assays described above may be used as initial or primary screens to detect promising lead compounds for further development. Often, lead compounds will be further assessed in additional, different screens. Therefore, this invention also includes secondary HYP modulators screens which may involve assays utilizing mammalian cell lines expressing HYP.

Tertiary screens may involve the study of the identified modulators in the appropriate rat and mouse models. Accordingly, it is within the scope of this invention to further use an agent identified as described herein in an appropriate animal model. For example, a test compound identified as described herein (e.g., a HYP inhibiting agent, an antisense HYP nucleic acid molecule, a HYP siRNA, a HYP antibody, etc.) can be tested in a transgenic mice overexpressing HYP of the present invention to determine the efficacy, toxicity, or side effects of treatment with such an agent. Furthermore, this invention pertains to uses of novel agents identified by the above-described screening assays for treatment of cancers, or other diseases characterized by unrestrained signalling through the RAS/MAPK pathway.

The present invention is illustrated in further details by the following non-limiting examples.

### Example 1

### CNK and KSR are Required for Activation of the Catalytic Domain of RAF

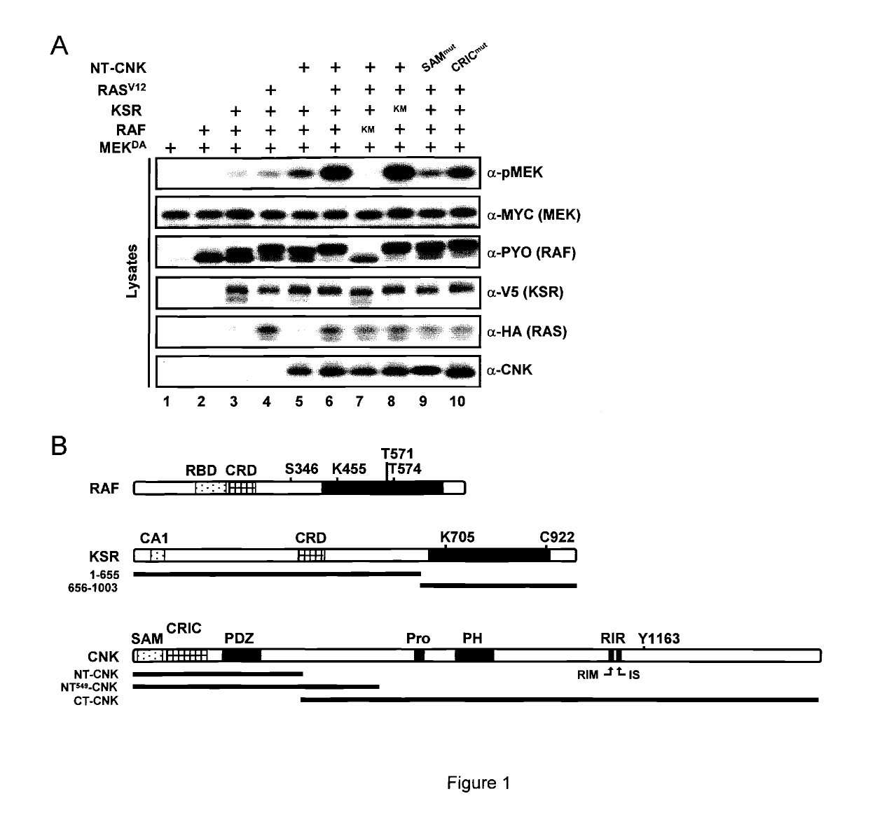

RAS-dependent activation of the MAPK module in Drosophila S2 cells was previously found to depend on two domains (SAM and CRIC) located in the amino-terminal portion of CNK (FIG. 1) and this requirement occurred at a step upstream of RAF (Douziech et al., 2003). This event was investigated at the molecular level, with a KSR-dependent MEK activation assay based on Drosophila proteins that was previously used to demonstrate KSR's ability to facilitate MEK phosphorylation by RAF (Roy et al., 2002). In that assay, co-expression of wild-type variants of epitope-tagged KSR, RAF and kinase-inactivated MEK^{DA}, is sufficient to induce MEK phosphorylation on activating residues S237 and S241 (FIG. 1, compare lanes 1-3), which could be further enhanced by co-expression of RAS^{V12} (lane 4). Interestingly, co-expression of NT-CNK (amino acid position 2-384; FIG. 1B) along with KSR, RAF and MEK, also enhanced MEK activation (lane 5), thus indicating that the effect of NT-CNK is integrated by the KSR/RAF/MEK complex. Consistent with the cooperation observed between RAS^{V12} and NT-CNK (Douziech et al., 2003), their co-expression with KSR, RAF and MEK resulted in a synergistic activation of MEK (compare lanes 4 and 6, or lanes 5 and 6). The specificity of this assay still critically depended on RAF catalytic function as a kinase-mutant RAF variant (RAF^{KM}) did not support MEK activation (lane 7). In contrast, although the presence of KSR was absolutely essential for MEK phosphorylation (see below), its function did not appear to depend on a putative kinase activity as a presumed catalytically-impaired mutant was as active as wild-type KSR (compare lanes 6 and 8, and see below). Finally, and most importantly, NT-CNK activity depended on the integrity of its SAM and CRIC domains (lanes 9 and 10).

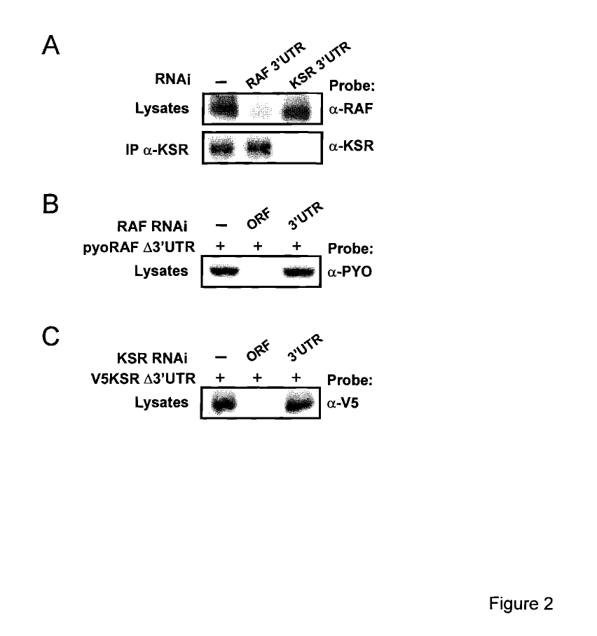

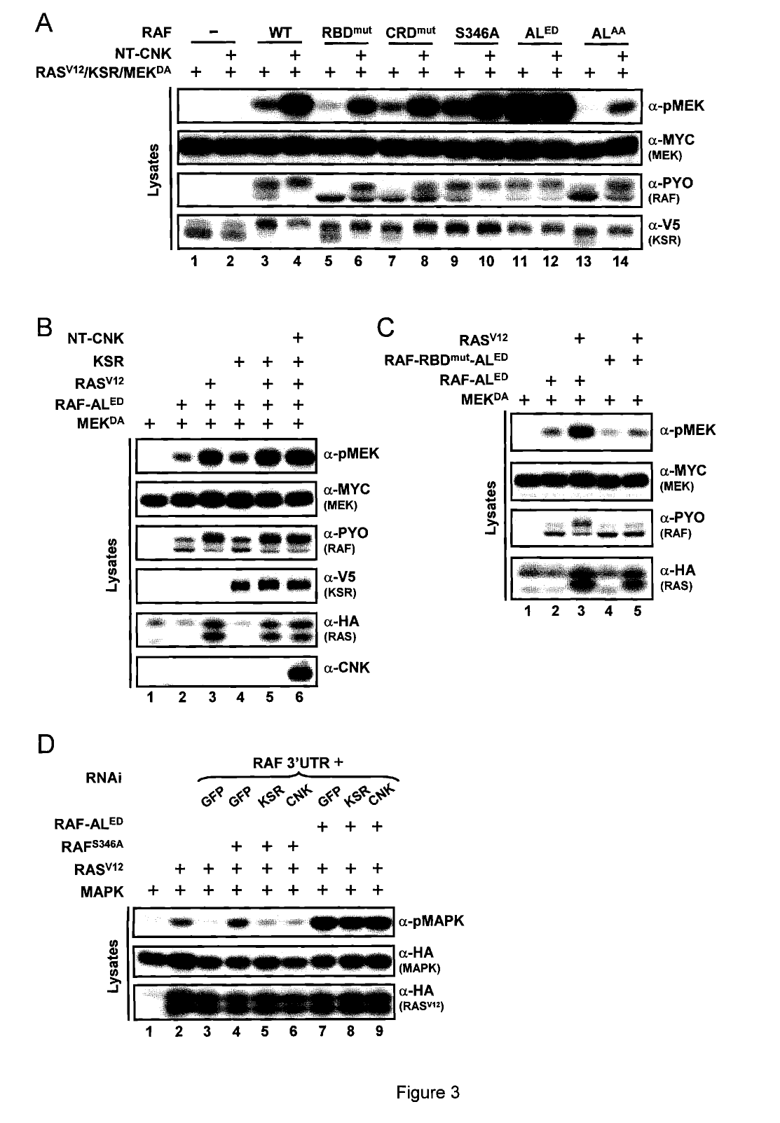

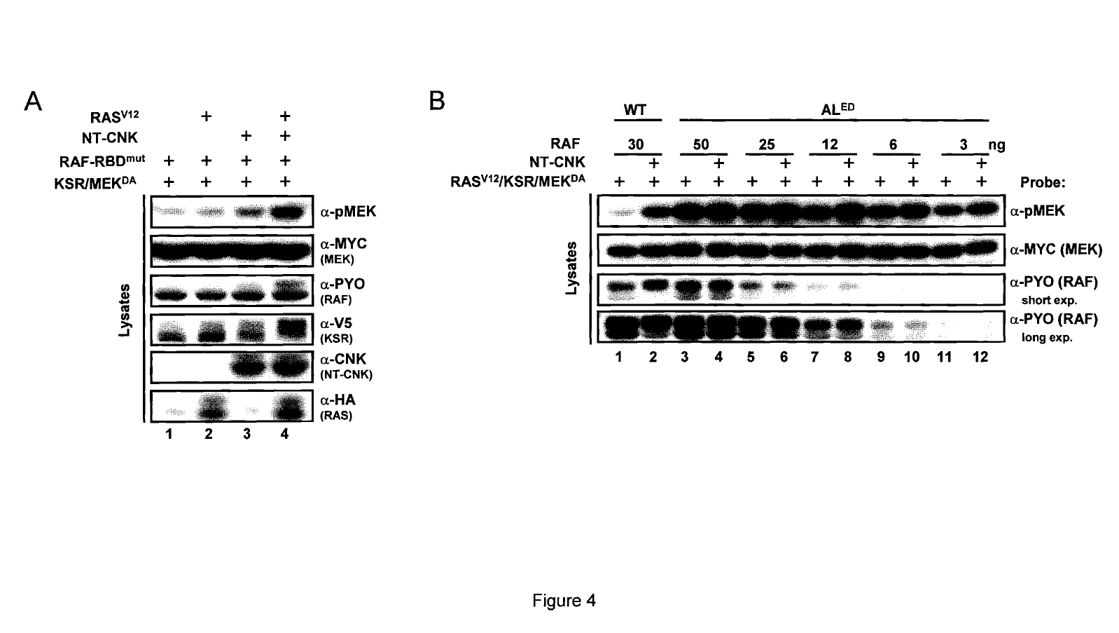

The assay presented above provides a molecularly tractable system enabling the more specific identification RAF regulatory event(s) that is/are positively modulated by CNK. Four regions within RAF known to carry out distinct function were thus independently mutagenized and the resulting response to NT-CNK was assessed. These regions included the RBD, the CRD, the S259-like residue (S346) within the N-terminal 14-3-3-binding site and the two phospho-accepting threonine residues (T571 and T574) within the activation loop of the kinase domain (FIG. 1B). To ensure that endogenous RAF did not influence the output, a RNAi-based strategy previously used to deplete the endogenous contribution of a given transcript while concomitantly restoring it exogenously using a non-targetable construct (Douziech et al., 2003) was used. Briefly, endogenous RAF was depleted (FIG. 2A) by transfecting a dsRNA encompassing exclusively RAF 3′UTR sequences and exogenously restored RAF activity by co-transfecting a RAF cDNA that had its 3′UTR sequences replaced by those of the Drosophila alcohol dehydrogenase (ADH) gene, thereby producing RAF transcripts insensitive to the dsRAF^{3UTR} RNA (FIG. 2B). As shown in FIG. 3A, co-transfection of RAS^{V12} with KSR and MEK^{DA} (−/+NT-CNK) in the absence of RAF did not induce MEK phosphorylation (lanes 1 and 2), while similar co-transfections that included wild-type RAF (WT) restored MEK phosphorylation (lane 3) as well as the response to NT-CNK (lane 4). Inactivation of the RBD, CRD and 14-3-3-binding site worked as expected: both the RBD and CRD mutations decreased RAF activity, whereas the S346A mutant was more active than WT (compare lanes 3, 5, 7 and 9). Interestingly, none of those variants impeded the response to NT-CNK (lanes 5-10), thus indicating that NT-CNK is probably not regulating the ability of the RAF N-terminal domain to associate with either RAS, the plasma membrane or 14-3-3. Nonetheless, it is remarkable that even though the RAF RBD^{RBDmut} variant no longer associates with RAS, the effect of NT-CNK on this mutant still depends on RAS^{V12} (FIG. 4A), thus further supporting the RAS-dependency of NT-CNK activity. Next, the effect of oppositely acting mutations within the activation loop (AL) of the kinase domain was evaluated. Mutations changing T571 and T574 to phospho-mimetic residues (AL^{ED}) resulted in a strongly activated RAF variant (lane 11). Surprisingly, this mutant no longer responded to NT-CNK (lane 12). To ensure that the levels of MEK^{DA} were not limiting under those conditions, similar experiments using decreasing amounts of RAF^{ED} we conducted and similar results were obtained (FIG. 4B). Conversely, changing the same threonine residues to alanine (AL^{AA}) produced a potent loss-of-function that yet retained some capacity to respond to NT-CNK (lanes 13 and 14). Taken together, these results suggest that NT-CNK activity controls a step leading to T571-T574 phosphorylation and that this activity either is distinct or has broader consequences than the phosphorylation event per se as the AL^{AA} mutant still responded to NT-CNK.

Whether the inability of RAF^{ED} to respond to NT-CNK was specific to CNK or whether this mutant also no longer integrated RAS and KSR activities was investigated. Interestingly, although RAF^{ED} activity could be further increased by RAS^{V12} (FIG. 3B, compare lanes 2 and 3), it was not influenced by KSR expressed either alone (lane 4) or in combination with RAS and/or NT-CNK (lanes 5 and 6, and data not shown). Given the presence of an intact RBD in RAF^{ED}, was verified whether it accounted for its responsiveness to RAS even if the RAS-dependent NT-CNK effect did not modulate RAF^{ED}. As shown in FIG. 3C, this appeared to be the case as a RAF^{RBDmut-ED} double mutant lost the capacity to respond to RAS^{V12} (lane 5). This finding therefore provides further evidence that the RBD-dependent and the NT-CNK-dependent steps are two independent RAS-mediated events. Curiously and in sharp contrast to RAF^{WT} or any of the other RAF mutants that has been tested so far, the fact that RAF^{ED} could not be modulated by KSR expression suggests that the RAF-MEK bridging effect of KSR is not critical for the ability of RAF^{ED} to phosphorylate MEK even if, as RAF^{WT}, RAF^{ED} does not stably associate with MEK (data not shown). Together, these results suggest that the scaffolding function of KSR is secondary to a more critical role played by KSR that influences the activation loop of RAF.

To determine whether endogenous CNK and KSR are also required at a similar step, their respective contribution by RNAi were depleted and the impact on RAF^{ED} activity was assessed using MAPK activation as readout. These experiments were conducted in the presence of RAS^{V12} so that appropriate RAS signals were provided to endogenous CNK and KSR. In addition, we depleted endogenous RAF by RNAi and restored it with similar amounts of the two distinct RAF gain-of-function tested above: RAF^{S346A a}nd RAF^{ED}. As shown in FIG. 3D, neither CNK nor KSR depletion reduced RAF^{ED} activity. In contrast, the activity of RAF^{S346A} was affected by the removal of endogenous CNK and KSR. Together, these results are consistent with a model whereby CNK and KSR activities are both required for events leading to the activated state of the RAF activation loop occurring downstream of the dephosphorylation of the S259-like residue.

### Example 2

### The Kinase Domain of KSR Mediates CNK Activity

Using a strategy analogous to the one used for RAF, it was next determined whether KSR is required for NT-CNK's positive function and if so, which structural feature of KSR was mediating the effect. Endogenous KSR was depleted by RNAi as in Example 1 (FIG. 2). First, a control experiment was conducted where RAS^{V12}, RAF^{WT} and MEK^{DA} were co-transfected in the absence of KSR to show its strict requirement for MEK phosphorylation (FIG. 4A, lane 1). Strikingly, co-transfection of NT-CNK under those conditions did not significantly elevate MEK phosphorylation nor did it increase RAF mobility shift (lane 2), thus demonstrating that KSR is essential for the positive effect of CNK on RAF. Indeed, introduction of KSR along with RAS^{V12}, RAF^{WT} and MEK^{DA} restored MEK activation (lane 3), which could be further enhanced by NT-CNK (lane 4). The impact of KSR mutants on the ability of NT-CNK to stimulate MEK phosphorylation was then tested. Two mutants that have either a decreased RAF binding (KSR^{CA1mut}) or a disrupted MEK binding (KSR^{C922Y}) activity were first evaluated. As previously shown (Roy et al., 2002), these mutations strongly reduced KSR activity (lanes 5 and 7). Interestingly, although the absolute output of pMEK was weaker than for KSR^{WT}, they were nonetheless capable of supporting NT-CNK activity (lanes 6 and 8). These results are especially surprising for KSR^{C922Y} as this variant totally lost its capacity to interact with MEK (Roy et al., 2002; see below). Therefore, these findings suggest that NT-CNK depends on a functional aspect of KSR that is distinct from its scaffolding property. To roughly delineate the region of KSR that is integrating NT-CNK function, the ability of the N-terminal portion (KSR¹⁻⁶⁵⁵; FIG. 1B) or the C-terminal portion (KSR⁶⁵⁶⁻¹⁰⁰³) of KSR to mediate NT-CNK effect were tested. Strikingly, the isolated kinase domain of KSR, albeit weakly, was capable of integrating NT-CNK function (compare lanes 11 and 12), whereas the N-terminal domain was totally inert (lanes 9 and 10), thus suggesting that NT-CNK effect is integrated by the kinase domain of KSR.

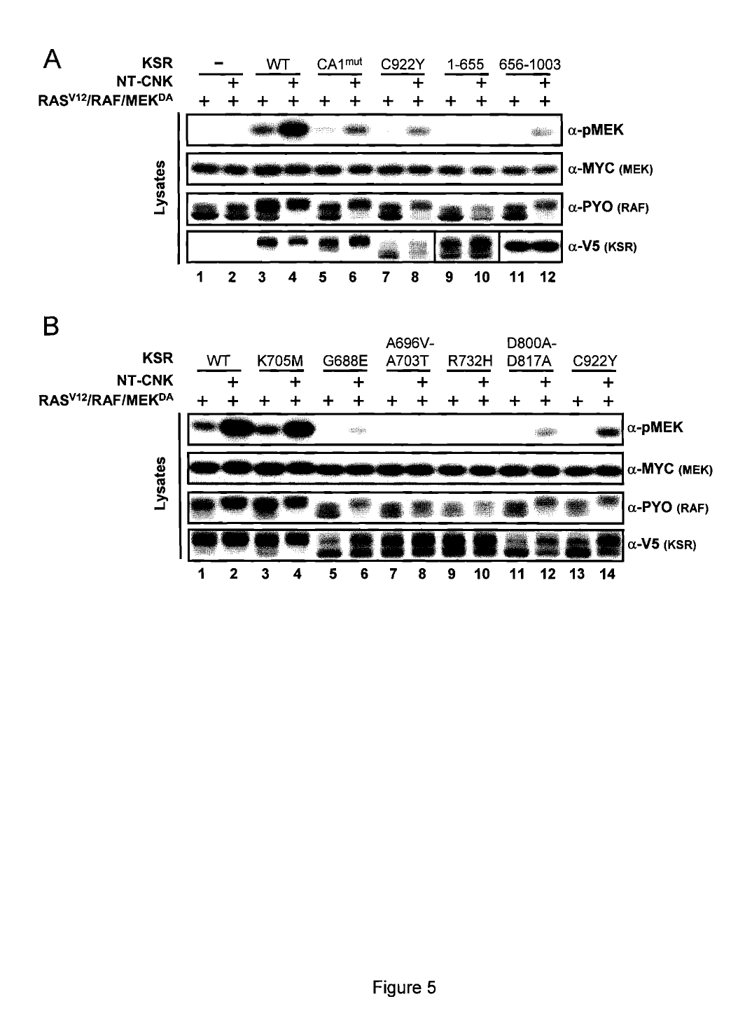

To further explore the relevance of the kinase domain of KSR in mediating CNK's positive function, the activity of a panel of kinase domain mutants were compared. As shown above, replacement of the typically critical lysine residue (KSR^{K705M}) located in subdomain II did not perturb KSR's ability to integrate NT-CNK activity (FIG. 5B, lanes 1 to 4), which again indicated that the positive effect of KSR and CNK on RAF does not involve standard kinase activity. In support for this conclusion, it was found that KSR^{D800A-D817A}, another catalytically-impaired mutant, retained the ability to respond to NT-CNK (lanes 11 and 12), even though it behaved as a strong loss-of-function given its disrupted MEK-binding activity (Roy et al. 2002; see below). In addition to the C922Y mutation, a number of point mutations located in the putative kinase domain have been recovered from genetic screens in Drosophila and C. elegans (Kornfeld et al., 1995; Sundaram and Han, 1995; Therrien et al., 1995). Some of these mutations were tested to determine whether any of them attenuated the response to NT-CNK. They included the G688E mutation and the A696V-A703T double point mutation that changed highly conserved residues within subdomain I (ATP-binding site) and sub-domain II, respectively, as well as the R732H mutation that affects a relatively well-conserved residue located between subdomains III (αC helix) and IV. Interestingly, although KSR^{G688}E retained some capacity to respond to NT-CNK (lanes 5 and 6), and thus behaved similarly to KSR^{C922Y} and KSR^{D800A-D817A}, the two other mutants (KSR^{A696V-A703T} and KSR^{R732H}) were totally inert (lanes 7 to 10). The failure of the A696V-A703T or R732H mutations to mediate NT-CNK effect suggests that they disabled an essential aspect of KSR function, thereby preventing any KSR-mediated activity.

### Example 3

### KSR is More than a Scaffold Connecting MEK to RAF

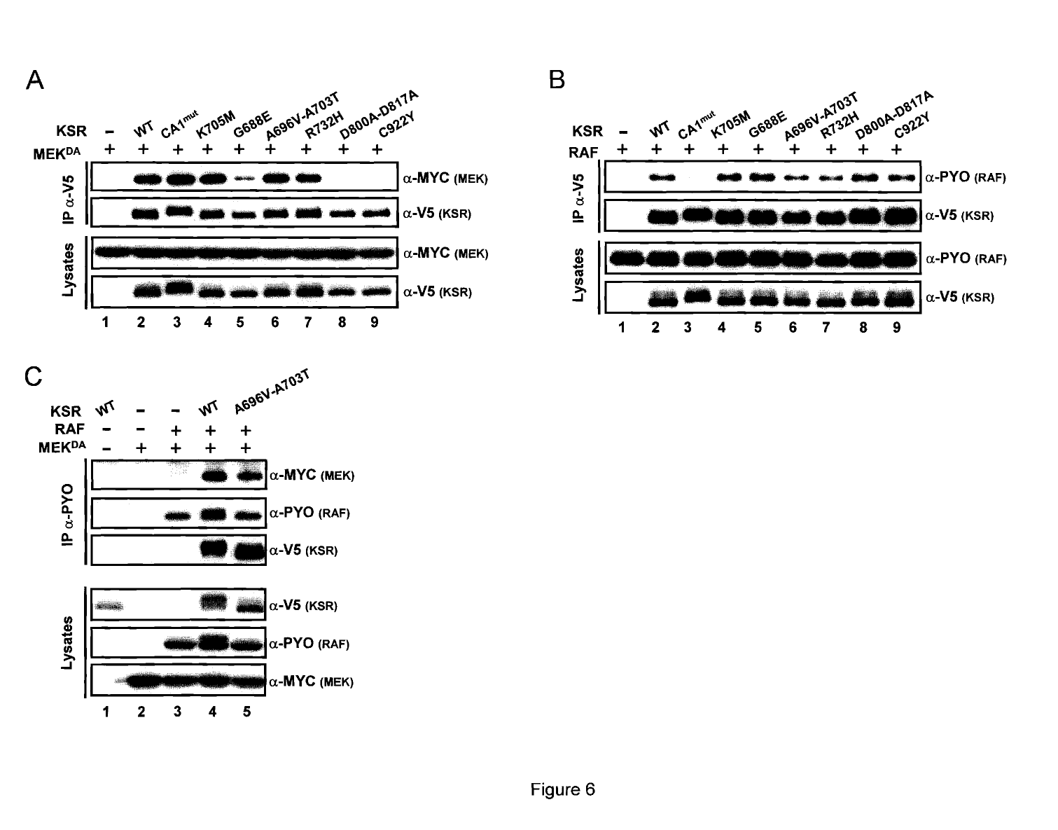

Mutations in KSR disrupting MEK or RAF binding, such as KSR^{CA1mut}, KSR^{C922Y} or KSR^{D800A-D817A} had been previously shown to impair KSR activity and were actually used as evidence to argue that KSR has a scaffolding role within the MAPK module (Roy et al., 2002). Compared to KSR^{WT}, KSR^{G688E} also displayed lowered MEK binding activity (FIG. 6A, compare lanes 2 and 5), but interacted normally with RAF (FIG. 6B, compare lanes 2 and 5). The reduced association of KSR^{G688E} to MEK is thus consistent with its loss-of-function behavior, although it is surprising that it is not more active than the two KSR mutants (KSR^{D800A-D817A} and KSR^{C922Y}) that entirely lost MEK binding (FIG. 6A, lanes 8 and 9). Unexpectedly, the two strongest mutants, KSR^{A696V-A703T} and KSR^{R732H}, showed normal association with either MEK (FIG. 6A, lanes 6 and 7) or RAF (FIG. 6B, lanes 6 and 7) and thus their inactivity could not be explained by a lack of MEK or RAF binding. Another property of KSR is its ability to bridge RAF and MEK. this ability was verified and no evidence was found that these two mutants are less competent than KSR^{WT} to induce a RAF/MEK complex (FIG. 6C and data not shown). Together, these findings demonstrated that the mere bridging of RAF and MEK by KSR is not sufficient for RAF to phosphorylate MEK and thus KSR must control another event. A number of scenarios can be envisioned to explain these results. For example and without being limited to such hypothesis, in addition to link RAF and MEK together, KSR may be involved in properly orienting MEK with respect to RAF, so that phosphorylation could normally proceed. However, such a model would not suffice to explain why RAF^{ED} does not respond to KSR. A second possibility, which would be consistent with the RAF^{ED} results, would be that KSR also controls RAF catalytic function.

### Example 4

### CNK Links KSR to RAF

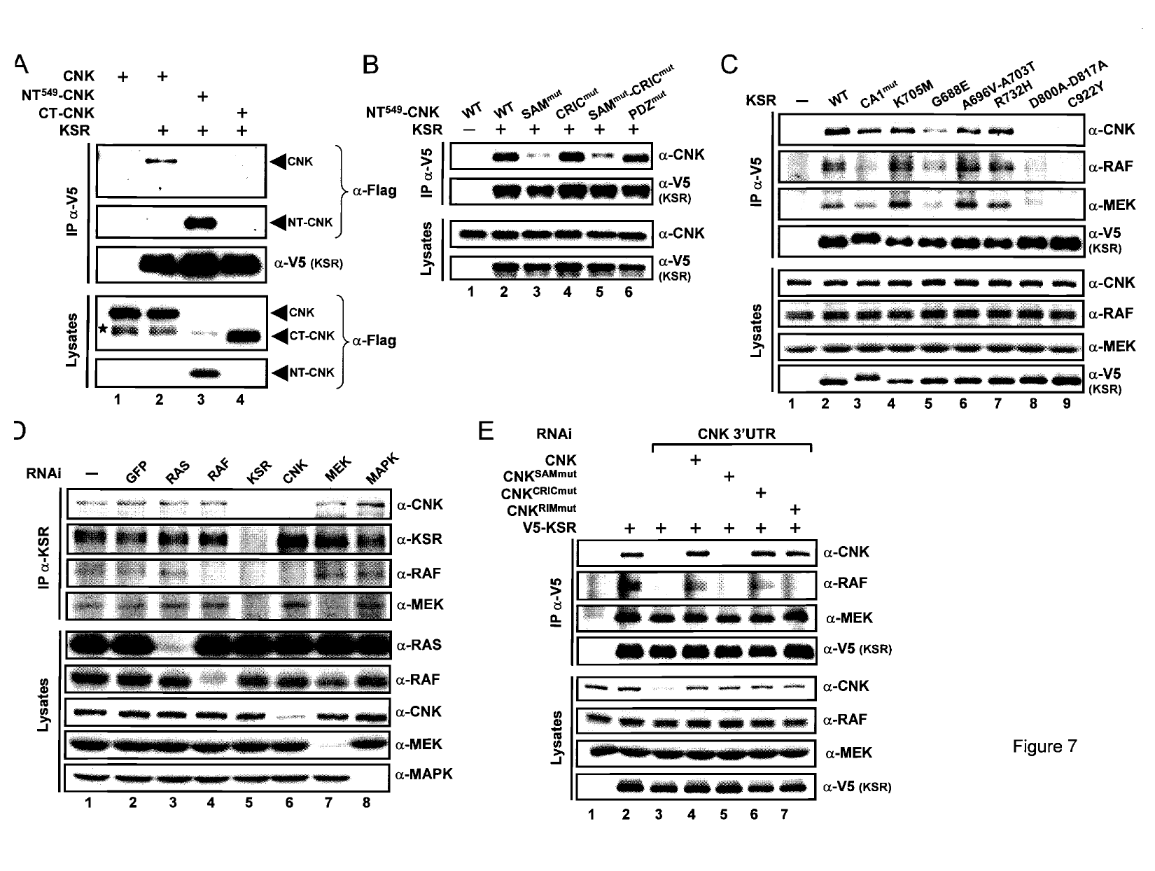

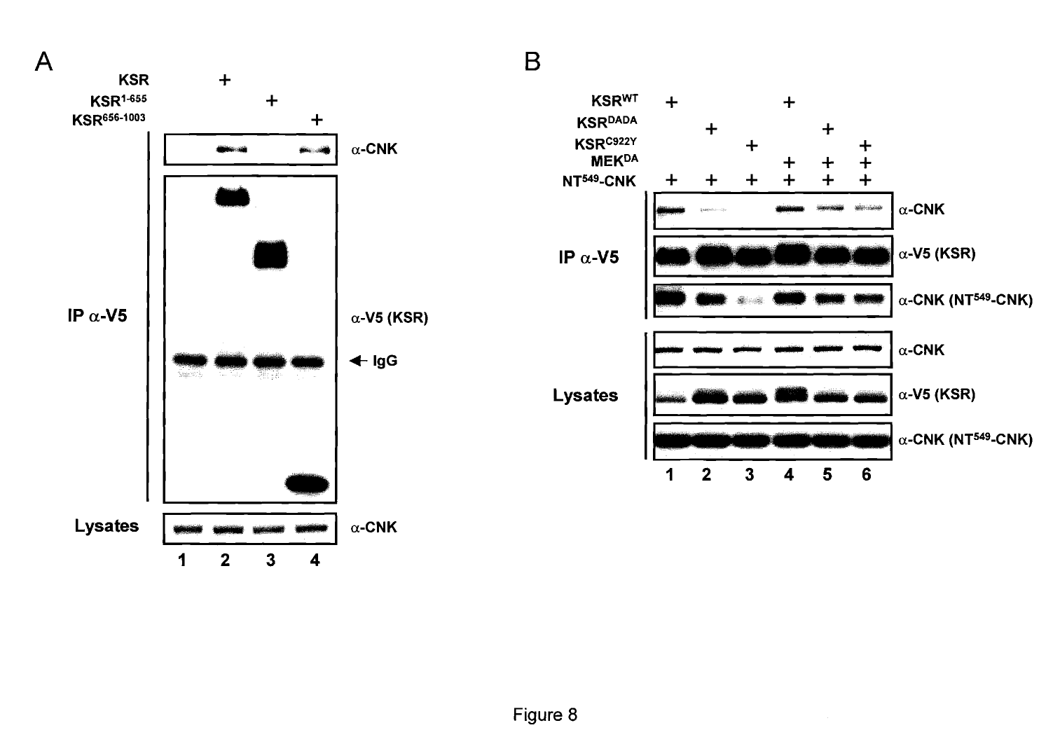

The results shown above suggest that CNK and KSR positively regulate RAF at a similar step and that CNK could not stimulate RAF activity without KSR. Based on this functional dependency, the possibility that CNK and KSR physically interacted was investigated. To verify this, Flag-tagged CNK and V5-tagged KSR were co-expressed in S2 cells, KSR was immunoprecipitated from cell lysates and the presence of CNK was assessed in the immunoprecipitate. As shown in FIG. 7A, it was found that CNK and KSR indeed associated when co-expressed together (lane 2). Given that both proteins can also interact with RAF, a similar experiment was conducted under conditions whereby endogenous RAF had been depleted by RNAi A to determine whether it mediates the CNK/KSR association, but no evidence was found for this scenario (data not shown and see below). To narrow down the region of CNK involved in this association, the ability of an N-terminal (NT⁵⁴⁹-CNK) and a C-terminal (CT-CNK) CNK construct (FIG. 1B) to co-immunoprecipitate with KSR was tested. These experiments showed that only the N-terminal region of CNK associated with KSR (FIG. 7A, lanes 3 and 4), thus indicating that a KSR-binding site resides in this area. Similar results were obtained with the shorter NT-CNK variant used in the functional studies, although that variant migrated immediately below the immunoglobulin heavy chain and thus was partly masked (data not shown). To determine if any of the three conserved domains of the N-terminal region of CNK is required for the association with KSR, the effect of individual point mutations inactivating each of these in the NT⁵⁴⁹-CNK context were tested. Markedly, inactivation of the SAM domain resulted in a severe loss of KSR binding (FIG. 7B, lane 3), whereas the CRIC or PDZ domain mutations did not alter the association (lanes 4 to 6). It was next sought to determine which region of KSR interacted with CNK. During the course of this work, it was noticed that expression of V5-KSR in S2 cells associated with endogenous CNK, RAF and MEK as determined by co-immunoprecipitation and immunoblot analysis (FIG. 7C, lane 2; and data not shown). The isolated N- and C-terminal domains of KSR were thus tested for their ability to associate with endogenous CNK. Consistent with the fact that NT-CNK activity is integrated by the kinase domain of KSR, it was found that endogenous CNK specifically associated with the C-terminal KSR construct (FIG. 8A). Similar results were obtained with Flag-tagged CNK or NT-CNK constructs (data not shown). Taken together, these results identify the SAM domain of CNK as a KSR-binding interface and thus provide a molecular link to explain the functional dependency of NT-CNK toward KSR.

Given the ability of V5-KSR to associate with endogenous CNK, RAF and MEK (FIG. 7C, lane 2), the panel of KSR kinase domain mutants was used to determine which residues are critical for those associations. As expected, the CA1 mutant only affected the association with endogenous RAF (FIG. 7C, compare lanes 2 and 3). Interestingly, the mutants that disturbed MEK binding (i.e. KSR^{G688E}, KSR^{D800A-D817A} and KSR^{C922Y}) also reduced the association with CNK (FIG. 7C, lanes 5, 8 and 9), thus suggesting that MEK and CNK binding on KSR depend on a common region/architecture of the putative kinase domain. Nonetheless, the fact that two of these mutations (D800A-D817A and C922Y) strongly affected endogenous CNK binding and yet responded to NT-CNK (FIG. 5B) appeared intriguing. It was thus verified whether NT-CNK could still associate with these two mutants and found that although the interactions were decreased, there was significant binding especially in a context of MEK overexpression (FIG. 8B, and data not shown), thus explaining the responsiveness of these mutants to NT-CNK. Another interesting observation was that the two most severe mutants, KSR^{A696V-A703T} and KSR^{R732H}, interacted normally with CNK as well as with MEK and RAF (FIG. 7C, lanes 6 and 7), therefore ruling out a binding defect with these proteins. Finally, and most surprisingly, the mutations affecting MEK and CNK binding also impeded endogenous RAF binding (lanes 5, 8 and 9), which contrasts with the KSR/RAF co-overexpression results shown in FIG. 6B. It was thus suspected that there could be more than one site of interaction between KSR and RAF and that the one present on the kinase domain of KSR is not direct, but depends on an endogenous protein. Given their similar binding profiles, endogenous CNK and/or MEK could be acting as mediators of the KSR/RAF association. Before testing this hypothesis, it was determined whether a KSR/CNK/RAF/MEK complex genuinely existed in vivo. To accomplish this, endogenous KSR were immunoprecipitated from plain S2 cells and the immunoprecipitates probed for the presence of endogenous CNK, RAF and MEK. As shown in FIG. 7D, the KSR antibody indeed brought down all four proteins (lane 1), whereas elimination of endogenous KSR by RNAi prevented their detection (lane 5), hence demonstrating the presence of such a complex in S2 cells. This immunoprecipitation procedure was then used with RNAi-treated cells to ask whether endogenous RAS activity is required for complex formation and if any of its known constituents mediates some of the associations. Depletion of endogenous RAS did not alter the ability of KSR to associate with CNK, RAF and MEK (lane 3), thus indicating that complex formation pre-exists to RAS activation. No effect also was detected when MAPK was depleted (lane 8). Besides abrogating their own presence in the complex, depletion of endogenous RAF or MEK did not alter the association of the other members of the complex (lanes 4 and 7). In contrast, depletion of endogenous CNK eliminated the association of RAF to KSR. Taken together, these results strongly suggest that MEK and CNK interact independently with KSR, whereas the KSR/RAF association is mediated by CNK, which would explain why mutations in KSR that impede CNK binding also affect endogenous RAF binding.

It was next sought to determine which domain(s)/element(s) in CNK are mediating the KSR/RAF association. To experimentally manipulate the CNK-dependent KSR/RAF association, endogenous CNK were depleted by RNAi and its presence rescued by transfecting non-targetable CNK constructs. As shown in FIG. 7E, depletion of endogenous CNK prevented co-immunoprecipitation of endogenous RAF with V5-KSR (compare lanes 2 and 3, panel α-RAF), whereas transfection of a wild-type CNK construct restored the interaction (lane 4). As predicted from the above binding studies, a rescue construct carrying a SAM domain mutation was incapable of restoring endogenous RAF binding as it failed to associate with V5-KSR (lane 5, α-RAF and α-CNK panels, respectively), thereby identifying the SAM domain as one of the critical points of contact to bridge KSR and RAF. On the other hand, a loss-of-function mutation within the CRIC domain did not prevent the formation of the KSR/RAF complex, therefore suggesting that the positive action of the CRIC is distinct from KSR/RAF complex formation. It was previously showed that the kinase domain of RAF binds a short sequence motif in CNK named the RAF-interacting Motif (RIM; Douziech et al., 2003). It was thus verified whether this element is involved in the KSR/RAF interaction. Strikingly, a CNK rescue construct carrying a mutation in this element completely failed to restore the KSR/RAF association, even though it interacted normally with KSR (lane 7). Together, these findings provide strong evidence that the SAM domain and the RIM element are, respectively, specific bridging sites for KSR and RAF and thereby enable the assembly of a KSR/RAF complex.

If the ability of CNK to bring together KSR and RAF is functionally relevant for the pathway, then one would predict that both the SAM domain and the RIM element are positively required for normal CNK function. While this appears to be the case for the SAM domain (FIG. 1A and Douziech et al., 2003), it is less clear for the RIM. Indeed, this element was initially defined as part of the so-called RAF Inhibitory Region (RIR; Douziech et al., 2003), which together with the IS element is essential for CNK's inhibitory effect on RAF. For instance, unlike wild-type CNK, shorter versions of CNK that do not contain these two motifs or full-length RIR-inactivated mutants have been found to strongly collaborate with RAS when tested by overexpression in S2 cells or during Drosophila eye development (Douziech et al., 2003; and FIG. 1). In vivo complementation assays were recently conducted using non-overexpressed genomic rescue constructs to show that the IS element of CNK has a genuine negative role with respect to RAF and that the RTK-dependent binding of Src42 to a C-terminal binding site relieves its inhibitory effect (Laberge et al., 2005). Moreover, a construct where only the IS element is disabled also fully restored the viability of cnk loss-of-function alleles, thus indicating that it does not fulfill a positive role.

To determine whether the RIM plays a positive role in a non-overexpressed context, a cnk genomic rescue construct containing a disrupted RIM element was tested for its ability to prevent the lethality associated with cnk loss-of-function. Mutations affecting either the SAM or the CRIC domains were also tested. In marked contrast to wild-type or IS-disrupted cnk constructs, a RIM-disrupted construct failed to complement cnk recessive lethality (Table 1 below). Similar findings were made with a double mutant construct that affected both the RIM and the IS elements, thus indicating that inactivation of the RIM is disrupting a positive function that cannot be restored by de-activating the IS element. As expected, mutations targeting either the SAM or the CRIC domains were also unable to reverse cnk lethality. Together, these results strongly suggest that like the SAM and CRIC domains, the RIM is fulfilling an essential positive role in physiological conditions, which would be consistent with its ability to juxtapose RAF to CNK-bound KSR. Such a positive role for the RIM is not incompatible with its negative property. Indeed, the RIM can be viewed as a mere binding site for RAF which, on the one hand, allows the formation of a KSR/RAF complex and, on the other hand, enables the IS element to block RAF activity in the absence of proper RTK-induced signals. This scenario would also explain why several cnk loss-of-function alleles recovered in the KSR-dependent genetic screen have molecular lesions producing mutant proteins similar to NT-CNK and yet behaved as loss-of-functions (Therrien et al., 1998; Laberge et al., 2005). It is interesting however that when overexpressed, NT-CNK (or related CNK variants that lacked a functional RIM) is collaborating with RAS to mediate KSR activity even though it does not bridge KSR and RAF. Again, a number of models could explain this observation. The one that is favor herein without being so limited is that CNK does not only connect KSR to RAF, it is also controlling the RAF-activating property of KSR in a RAS-dependent manner. Therefore, when NT-CNK is overexpressed in the presence of activated RAS, a greater proportion of endogenous KSR become activated. As these activated KSR molecules still retain one physical contact to RAF through their CA1 region, the sum of their weaker effect results in non-negligible RAF activity.

TABLE 1
Genomic rescue experiments revealed the positive role played by the
RAF-Interacting Motif (RIM) of CNK

| | % of cnk^{E-1088}/ | |
|---|---|---|
| | cnk^{I(2)k16314} | # of flies |
| Genotype | adult flies^{a} | scored^{b} |
| cnk^{E-1088}/cnk^{I(2)k16314} | 0 | 1287 |
| cnk^{E-1088}/cnk^{I(2)k16314}, pcnk-cnk^{WI} | 21.9 | 515 |
| cnk^{E-1088}/cnk^{I(2)k16314}, pcnk-cnk^{ISmut} | 22.1 | 951 |
| cnk^{E-1088}/cnk^{I(2)k16314}, pcnk-cnk^{RIMmut} | 0.1 | 1215 |
| cnk^{E-1088}/cnk^{I(2)k16314}, pcnk-cnk^{RIMmut-ISmut} | 0 | 748 |
| cnk^{E-1088}/cnk^{I(2)k16314}, pcnk-cnk^{SAMmut} | 0 | 1167 |
| cnk^{E-1088}/cnk^{I(2)k16314}, pcnk-cnk^{CRICmut} | 0.3 | 309 |

Larval lethality associated with cnk^{E-1088}/cnk^{I(2)k16314} trans-heterozygous flies is rescued by introducing of one copy of a wild-type cnk genomic rescue construct (pcnk-cnk^{WT}).
^{a}For complete rescue, the expected ratio of cnk^{E-1088}/cnk^{I(2)k16314} adult flies over total progeny is 20%.
^{b}Results are shown for one transgenic line. Similar results were obtained with a second transgenic line for each construct.

### Example 5

### The KSR/CNK Interaction Depends on Hyphen, a Novel SAM Domain-Containing Protein

The present invention also related to the identification of the involvement of the SAM domain of CNK in the CNK/KSR interaction. Although initial studies on SAM domains revealed their ability to form homo- or hetero-oligomers with other SAM domains, mounting evidence suggests that they can also interact with unrelated proteins or even with RNA (Qiao et al., 2005). Hence, it is conceivable that the SAM domain of CNK directly contacts the kinase domain of KSR. However, it is also possible that the interaction is indirect and/or depends on additional components.

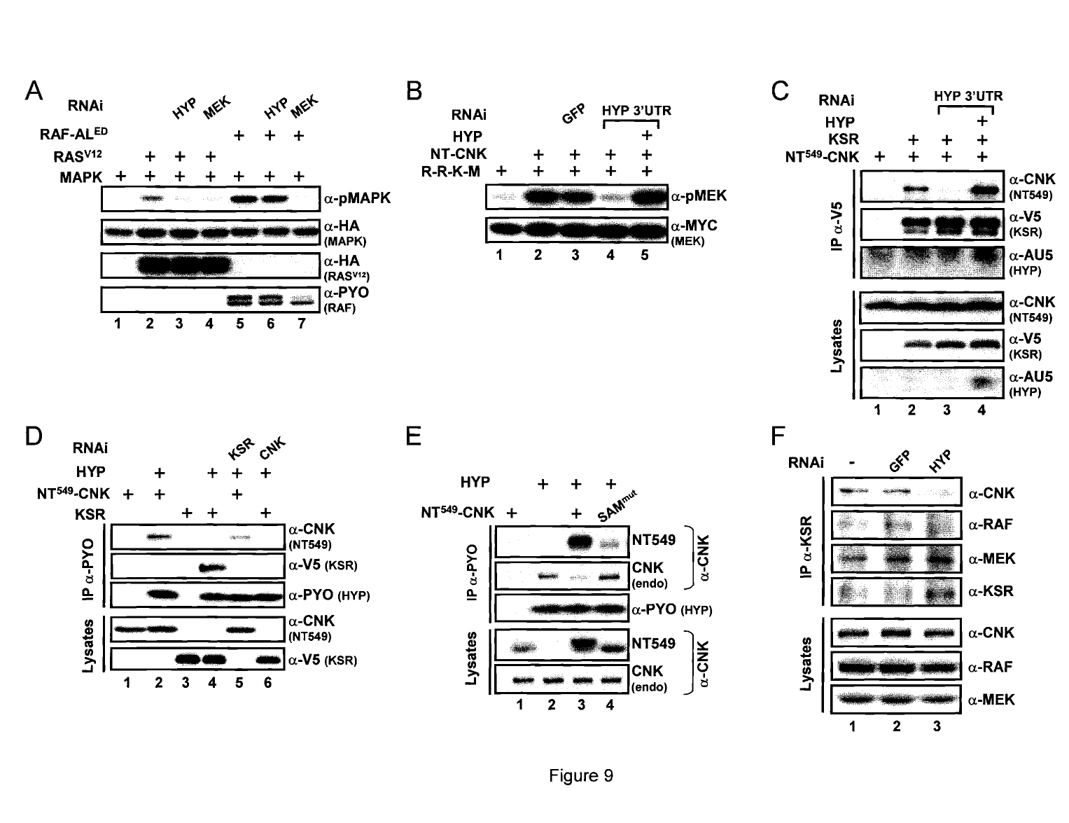

The present invention suggests that the SAM domain of CNK does not homodimerize (data not shown). Yet, it is surprising that the L71K mutation used herein and which is expected to preserve the structural integrity of the SAM domain while preventing it from oligomerizing (Stapleton et al., 1999), is as deleterious as a drastic mutation (W17S-I18S) predicted to unfold the domain (Douziech et al., 2003). This observation indicated that for proper function, the SAM domain of CNK possibly heterodimerizes with another SAM domain-containing protein. If this is the case, elimination of this hypothetical protein should impede RAS-mediated MAPK activation. To investigate this possibility, a selected set of SAM domain proteins found in Drosophila were targeted by RNAi and their effect tested on RAS-induced MAPK activation in S2 cells. In addition to CNK, Drosophila contains thirty-five independent SAM domain proteins according to the Single Modular Architecture Tool (SMART) database (Letunic et al. (2006) Nucleic Acids Res 34, D257-D260). Seven of these, which are transcription factors or modulators of transcription factors, contain a variant form of the SAM domain also known as the Pointed (PNT) domain (Slupsky et al., 1998) and were not considered further. Another nine were also not selected at this point because they corresponded either to chromatin-remodeling factors or RNA-binding proteins, or simply because available information did not suggest an involvement in RAS/MAPK signaling. Besides CNK, eighteen candidate proteins remained for testing (see Example 6 for corresponding CG numbers). Double-stranded RNA was thus produced for each of these and then tested separately. Interestingly, one of these dsRNAs, which targeted CG30476, significantly reduced MAPK activation induced by RAS^{V12} (FIG. 9A, compare lanes 2 and 3), while the others had no noticeable effect (data not shown). Moreover, in contrast to endogenous MEK depletion, elimination of CG30476 did not affect MAPK activity stimulated by RAF^{ED} (compare lanes 6 and 7), thus positioning its requirement upstream or in parallel to RAF. Therefore, these findings not only identified a potential candidate regulating the function of CNK, they also unveiled a novel mediator of RAS/MAPK signals.

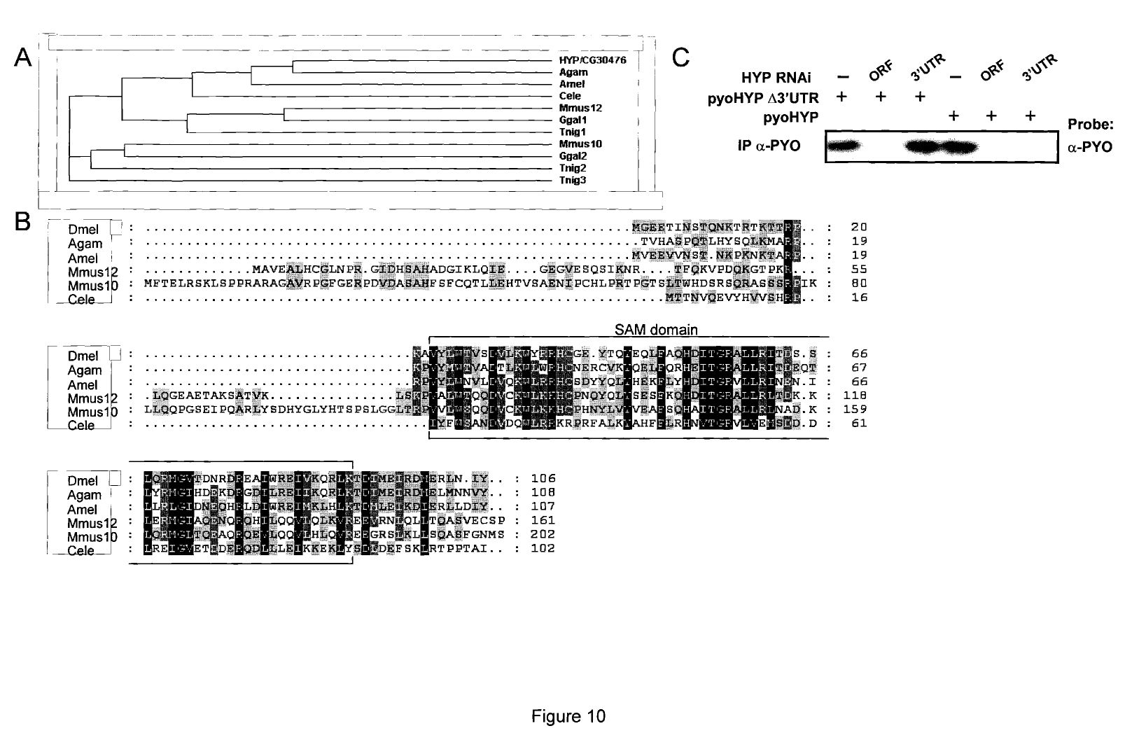

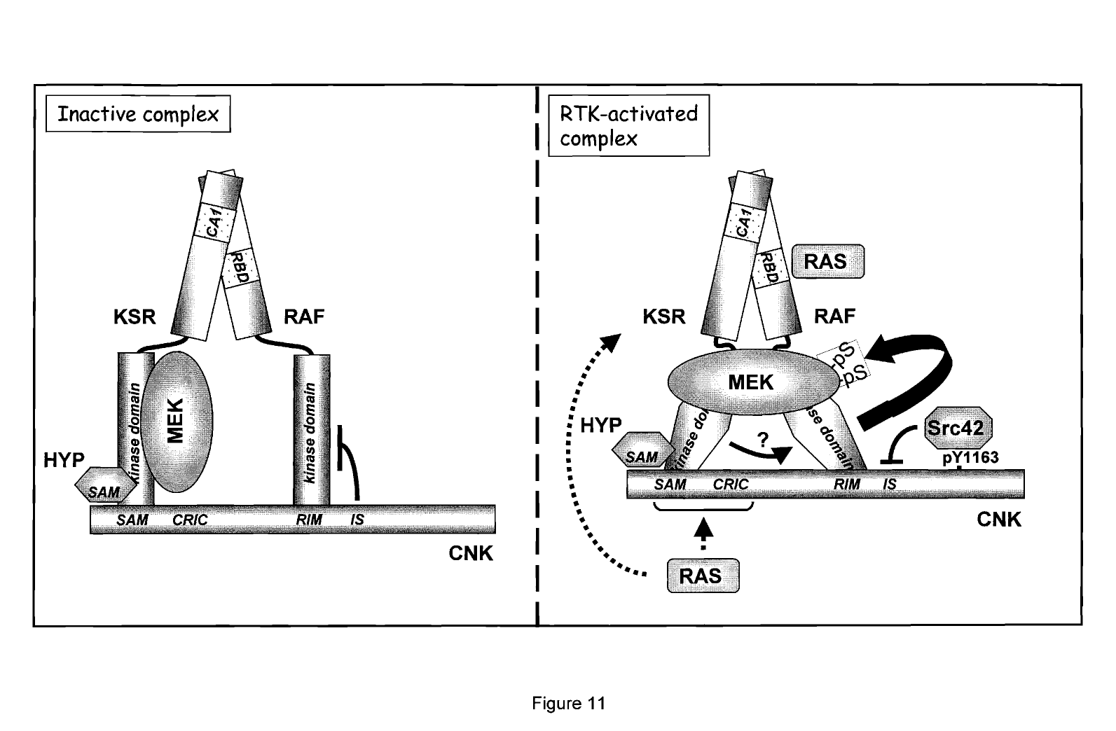

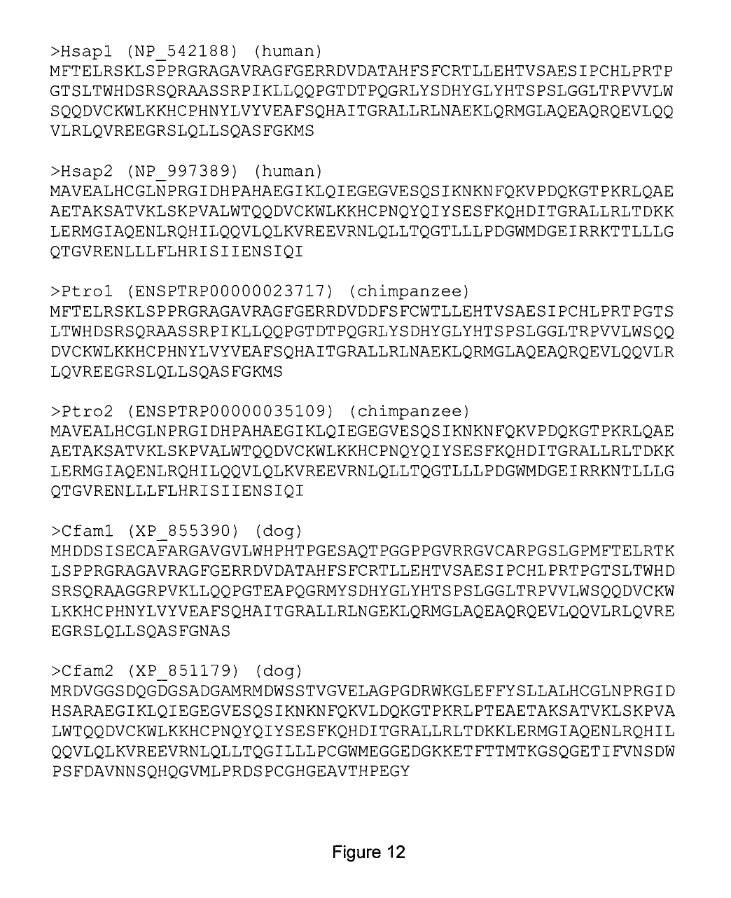

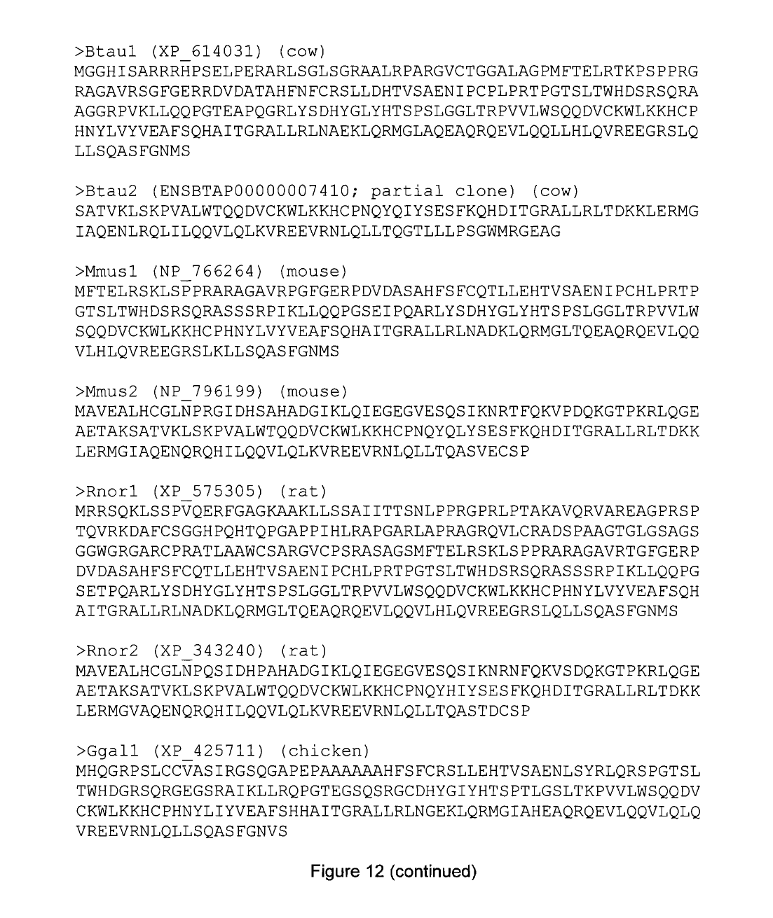

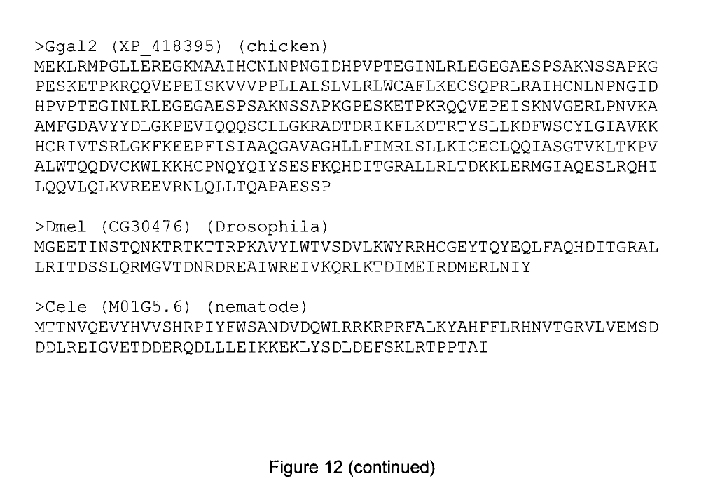

CG30476 is an evolutionarily conserved low molecular weight protein of 106 amino acids (SEQ ID NO: 15) from Drosophila that essentially consists of a single SAM domain followed by a short stretch of conserved residues (FIG. 10B and FIG. 13). This protein is identified herein Hyphen (HYP) based on its presumed ability to functionally link critical proteins within the RAS/MAPK pathway. If HYP is influencing the function of the SAM domain of CNK, then depletion of endogenous HYP should have an impact on NT-CNK-induced MEK activation. This is indeed what the present invention shows as RNAi-mediated HYP knock-down potently counteracted RAF-dependent MEK phosphorylation stimulated by RAS^{V12}, KSR and NT-CNK (FIG. 9B, compare lanes 2 and 4). The specificity of the RNAi was demonstrated by rescuing the MEK activation defect using a non-targetable HYP construct (lane 5; see also FIG. 10C). The relevancy of HYP for the KSR/CNK association was then tested. This point was first addressed by verifying whether the interaction between V5-KSR and NT-CNK⁵⁴⁹ depended on endogenous HYP. Strikingly, depletion of endogenous HYP by RNAi severely reduced the KSR/CNK interaction (FIG. 9C, compare lanes 2 and 3). Further, a HYP rescue protein that reversed the effect of HYP depletion (lane 4) could also be detected in the V5-KSR immunoprecipitate (lane 4; α-AU5 panel). Thus, these findings strongly suggest that HYP is essential for the formation of the KSR/CNK complex and that it is part of it.

Next, it was determined whether HYP interacts independently with KSR and CNK or whether its association with one of these two proteins enables the recruitment of the other. To verify this, HYP was co-expressed either with NT-CNK or with KSR and it was determined by immunoprecipitation whether HYP forms a complex with either protein. As shown in FIG. 9D, immunoprecipitation of PYO-tagged HYP brought down NT-CNK (lane 2) as well as KSR (lane 4). However, as endogenous KSR or CNK may participate in these binary interactions, these experiments were repeated in the presence of dsRNAs targeting endogenous KSR or CNK. Remarkably, while KSR RNAi only slightly decreased the HYP/CNK association (lane 5), removal of endogenous CNK totally eliminated the HYP/KSR interaction (lane 6). Therefore, these results suggest that the CNK/HYP association is a pre-requisite for KSR recruitment, although the presence of KSR may stabilize the CNK/HYP interaction.

The results presented thus far are compatible with a model whereby HYP associates with the SAM domain of CNK which in turn creates a binding interface for KSR. To verify the relevancy of the SAM domain of CNK for the CNK/HYP association, the ability of PYO—HYP to interact with wild-type and SAM^{mut} (L71K) NT⁵⁴⁹-CNK was compared by co-immunoprecipitation. As predicted, the SAM^{mut} variant had a much reduced capacity to interact with HYP (FIG. 9E; compare lanes 3 and 4), thus showing that the SAM domain of CNK is involved in the CNK/HYP association. A related observation also supported this conclusion. Indeed, it was found that PYO—HYP also associated with endogenous CNK (FIG. 9E, lane 2) and that this interaction could be competed by the presence of wild-type NT⁵⁴⁹-CNK, but not by the SAM^{mut} version (lanes 3 and 4).

Finally, it was tested whether HYP is genuinely required for the formation of the endogenous KSR/CNK complex observed in S2 cells. Endogenous KSR from S2 cells treated or not with HYP dsRNA were immunoprecipitated and the presence of associated CNK, RAF and MEK was assessed. Consistent with the above findings, reduction of endogenous HYP lowered the natural KSR/CNK interaction and as a result decreased the amount of associated RAF, but did not perturb the KSR/MEK interaction (FIG. 9F).

Taken together, these results identify HYP as a novel component of the RAS/MAPK pathway whose function, through its association with the SAM domain of CNK, enables the recruitment of KSR to CNK. Although the CNK/HYP binding mode and its structural consequence allowing KSR binding have yet to be determined, the findings of the present invention are intriguingly similar to the recruitment of Rolled/MAPK to YAN, a Drosophila SAM/PNT domain-containing transcriptional repressor that is inactivated following its phosphorylation by MAPK (Rebay and Rubin, 1995). In that particular case, the recruitment of MAPK to YAN depends on a short SAM domain protein, known as MAE, that heterodimerizes with the SAM domain of YAN (Baker et al., 2001).

### Concluding Remarks

The ability of KSR to promote the formation of RAF/MEK complexes independently of RAS signals was previously demonstrated and it was proposed that this scaffolding effect is a key functional aspect of KSR (Roy et al., 2002). The present invention showed that KSR does not act alone to bring RAF and MEK together, but requires at least two other proteins, namely, CNK and HYP (FIG. 11). Importantly, the data presented herein shows that CNK/HYP-bound KSR activates RAF in a RAS-dependent manner and that this function occurs at a step regulating the activation loop of RAF. Given that Drosophila KSR does not appear to have intrinsic kinase activity as mutagenesis of an essential residue for catalysis (i.e. K705M) still displays strong activity, it suggests that KSR does not phosphorylate the activation loop residues of RAF and thus either another kinase is recruited to accomplish this task or RAF itself is executing it.

Interestingly, CNK and HYP do not exhibit any positive activity unless KSR is present (FIG. 5 and data not shown), while KSR overexpression can induce RAF-mediated MEK phosphorylation independently of RAS and CNK (FIG. 1; Roy et al., 2002; and data not shown). It thus appears that KSR mediates the effect of RAS and CNK and that it can even bypass their requirement when expressed at sufficiently high levels as if it carries an intrinsic RAF-activating property that is unveiled when overexpressed along with wild-type RAF and MEK. Various models can be envisioned to explain the RAF-activating property of KSR. The one favored herein without being bound by such hypothesis is based on the position where KSR operates during this event and on the strong architectural and amino acid sequence homology between KSR and RAF members. A recent crystallographic study of the inactive B-RAF catalytic domain has uncovered an inhibitory interaction that takes place between the P loop and the DFG motif/activation loop (Wan et al., 2004). Structural analysis of this interaction strongly suggests that phosphorylation of the activation loop interferes with the interaction and thereby helps in switching and/or locking the DFG motif/activation loop into the active conformation. The importance of disrupting the inhibitory configuration is also strikingly suggested by the finding that up to 90% of a large number of B-RAF oncogenic mutations found in human melanomas affect a valine residue (V600) that stabilizes the inactive conformation (Davies et al., 2002; Wan et al., 2004). In fact, most of the other oncogenic B-RAF mutations recovered in melanoma cells could also be understood by their ability to disturb the inhibitory configuration. Surprisingly, some affected residues participating in catalysis and hence decreased intrinsic kinase activity. As these mutations were capable of elevating endogenous ERK activity by their ability to stimulate endogenous wild-type RAF proteins, it has been proposed that a catalytically impaired but conformationally derepressed RAF kinase domain transduces its effect to inactive RAF proteins, possibly via an allosteric process, and as a result promotes their catalytic activation. KSR may act through a similar mechanism. Its overexpression along with MEK and RAF may allow it to adopt a conformation that in turn disrupts the inhibited configuration of the RAF catalytic domain. This event would then position the activation loop of RAF in a suitable configuration for phosphorylation which ultimately stabilizes the catalytically activated state. In physiological conditions, KSR may also operate via this process, but presumably in a regulated manner (FIG. 11). For example, the conformation of the kinase domain of KSR might be controlled allosterically by the CNK/HYP complex in a RAS-dependent manner which in turn induces an activating conformational change in the kinase domain of RAF. This scenario might explain why the RAF-AL^{AA} mutant still responded to NT-CNK (FIG. 3A) as even if its activation state could not be stabilized by phosphorylation, its conformation might still be controllable allosterically thereby resulting in detectable catalytic activity. Although not mutually exclusive, KSR may also work by bringing other RAF-activating proteins or sequestering inhibitory proteins from RAF. The identification of two mutations (KSR^{A696V-A703T} and KSR^{R732H}) that completely eliminate the RAF-activating property of KSR, but that do not affect its RAF/MEK scaffolding function, should prove valuable to ascertain biochemically and structurally this novel function.

Collectively, the present characterization of CNK's functional elements/domains is providing novel insights as to how scaffold proteins can dynamically influence signaling within a given pathway. Indeed, it appears that prior to signal activation, the CNK/HYP pair juxtaposes a KSR/MEK complex to RAF and, owing to the Inhibitory Sequence (IS) of CNK, maintains this higher order complex in an inactive state by selectively repressing RAF catalytic function (Douziech et al., 2003; FIG. 11, left panel). Then, upon signal activation, CNK integrates two RTK-elicited signals that together leads to RAF activation (FIG. 11, right panel). First, RTK-induced phosphorylation of the Y1163 residue of CNK allows the binding of Src42, which in turn releases the inhibitory effect of the IS motif (Laberge et al., 2005). Second, RTK-induced RAS activity not only acts through the RBD of RAF, but also via the SAM-CRIC region of CNK (Douziech et al., 2003; data presented herein), thereby enabling KSR to activate RAF. How the N-terminal domains of CNK integrate RAS activity is currently unknown. One possibility is that the SAM domain, in association with HYP, merely acts as a binding interface for KSR, while the CRIC region is the one that perceives RAS activity and communicates it to KSR. It is also conceivable that RAS send signals to KSR independently of CNK and as a result allows KSR to respond to NT-CNK.

### Example 6

### Material and Methods

### Plasmids

Plasmids used in transfection experiments were all derived from the copper-inducible pMet vector (Therrien et al., 1998). Point mutations and deletions were generated by standard procedures and modified inserts were completely sequenced. Specific residues that were changed are indicated in the text or figure legends. CNK, KSR, RAF and HYP cDNA inserts ended at their respective Stop codon followed by the adh 3′UTR sequences so that they could be used in RNAi-based rescue experiments as previously described (Douziech et al., 2003).

NT⁵⁴⁹-CNK construct was made by introducing an L550Stop mutation in pMet-full length CNK. Other CNK constructs used in transfection experiments have been previously described (Douziech et al., 2003). For in vivo rescue experiments, assembly of a pBS-based (Stratagene) construct containing cnk genomic sequences was described in Laberge et al. (2005). Point mutations were separately introduced in this template (SAMmut [L71K]; CRICmut [del A162-H163-R164]; RIMmut [T1068A-L1069A-K1070A]; ISmut [G1092A-V1093A-E1094A] or double RiMmut-ISmut) and mutant inserts were then transferred to a pCaSpeR P-element vector (Pirrotta, 1988).

KSR constructs contained a V5 epitope (GKPIPNPLLGLDST (SEQ ID NO: 37)) at their N-terminus, while RAF constructs contain two PYO (Glu) epitopes (MEYMPME (SEQ ID NO: 38)). Hyphen full-length cDNA was amplified by PCR from the LD cDNA library (Berkeley Drosophila Genome Project). Two AU5 epitopes (TDFYLK (SEQ ID NO: 39)) or two PYO epitopes were introduced at its N-terminus. pMet-haRAS^{V12} and pMet-mycMEK^{DA} were described in Roy and al (2002) while pMet-haMAPK was presented in Douziech et al. (2003).

Cell Transfection, RNAi, cell Lysates and Immunoprecipitations

S2 cells were maintained in FBS-containing Schneider (Invitrogen) or serum-free insect cell media (Sigma). For transfection experiments, 7×10⁶ cells were plated per 60 mm dish and transfected the next day with various plasmid combinations±specific dsRNAs (1 μg total of nucleic acids) using the Effectene™ transfection reagent (Qiagen). Protein expression was induced by adding CuSO₄ [0.7 mM] to the medium at 18-24 h post-transfection. Double-stranded RNA production for RNAi experiments were produced and used in transfection experiments essentially as described in Roy et al. (2002). The eighteen SAM domain-containing proteins selected for RNAi were: CG4719, CG5272, CG7915, CG9098, CG9126, CG10743, CG11199, CG11206, CG12424, CG13320, CG13859, CG13996, CG15625, CG16757, CG18543, CG30476, CG31163, and CG31187.

To prepare protein lysates, cells were harvested 36 h post-induction in ice-cold PBS and then incubated for 15 min on ice in 500 μl of lysis buffer (20 mM Tris pH 8.0, 137 mM NaCl, 10% glycerol, 1% Igepal CA-630, 1 mM EDTA, 1 mM phenylmethylsulfonyl fluoride, 1 mM Na₃VO₄, 0.15 U/ml aprotinin, 20 μM leupeptin). Cell debris were removed by centrifugation at 12,000 g for 15 min (4° C.).

For immunoprecipitations, antibodies and protein A/G Plus™-agarose beads (Santa Cruz Biotechnology, Inc.) were added together to cell lysates (˜2 mg total proteins) and gently rocked for 4 h at 4° C. Immunoprecipitated proteins were then washed three times with cold lysis buffer before analysis.

### Western Blot Analysis and Antibodies

For immunoblot analysis, cell lysates and immunoprecipitated proteins were resolved on 8%-10% SDS-PAGE and transferred to nitrocellulose membranes. Sources for α-CNK, α-MAPK, α-pMAPK, α-MEK, α-pMEK, α-RAF, α-HA, α-MYC and α-PYO antibodies were mentioned in Douziech et al. (2003). Monoclonal α-V5 and polyclonal α-AU5 antibodies were obtained from Invitrogen and Bethyl Laboratories, respectively, whereas the α-KSR monoclonal antibody was described in Therrien et al. (1996).

### Genetics

Fly husbandry, crosses and P-element-mediated germline transformations were conducted according to standard procedures.

### Example 7

### Mammalian Hyphen Homologues are Involved in RAS-Mediated MAPK Activation

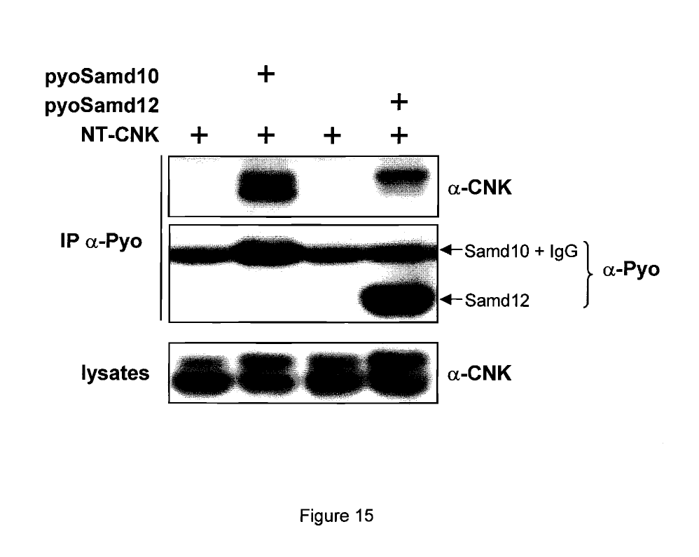

To determine whether mammalian homologues of Drosophila Hyphen function similarly to Hyphen, the mouse isoforms (Samd10[Genbank accession # AAH19475] and Samd12 [Genbank accession # AA119384]) were tested to determine whether they could associate with Drosophila CNK as does Drosophila Hyphen. To accomplish this, pyo-tagged mSamd10 or pyo-tagged mSamd12 were co-expressed with Flag-tagged N-terminal CNK (residues 2 to 384, which includes the SAM domain of CNK) in Drosophila S2 cells and the Hyphen homologues were immunoprecipitated using a Pyo antibody. As shown in FIG. 15, NT-CNK could be immunoprecipitated by both isoforms, thus indicating the formation of a stable complex between Drosophila CNK and the mouse Hyphen homologues.

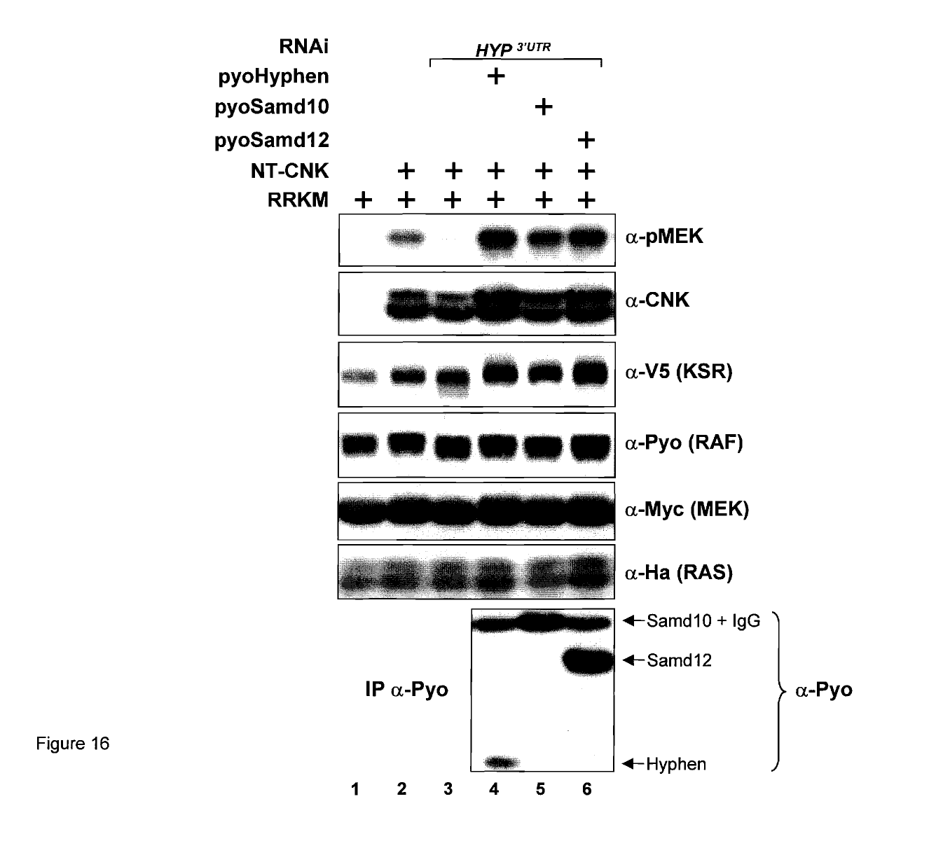

It was next evaluated whether the mouse homologues could replace Drosophila Hyphen within the Drosophila RAS/MAPK pathway. As shown above, co-expression of activated RAS with a CNK-assembled KSR/RAF/MEK complex strongly leads to MEK activation and this mechanism depends on the presence of endogenous Hyphen (see FIG. 16, compare lanes 1, 2 and 3). Using an RNAi-based assay that allowed the specific removal of endogenous Hyphen in S2 cells, it was tested whether re-introduction of either Samd10 or Samd12 could restore MEK activation. As shown in FIG. 16, both homologues were as competent as a Drosophila Hyphen construct to restore MEK activation (compare lanes 3 to 6).

Together, these results suggest that the mammalian Hyphen homologues also have the capacity to heterodimerize with the SAM domain of CNK through their respective SAM domain and as a consequence lead to the recruitment of a KSR/MEK complex enabling RAF activation.

The comparison of the amino acid sequence of mammalian Hyphen homologues with that of Drosophila Hyphen indicate which amino acids of the amino acid sequence of members of the Hyphen family can be substituted without abrogating their activities in the RAS/MAPK pathway. Hence, it is submitted that the amino acids that are not conserved between species can be replaced by other amino acids without abrogating the above disclosed Hyphen activity.

Certain substitutions are preferred however and correspond to either a substitution by an amino acid having similar chemical properties or, even more preferred, a substitution by the amino acid found at the corresponding position in Hyphens of other species. Amino acids are categorized herein into 5 groups of amino acids according to their chemical properties, namely small nonpolar (i.e. C, P, A and T), small polar (i.e. S, G, D and N), large polar (i.e. E, Q, K and R), intermediate polarity (i.e. Y, H and W), and large nonpolar (i.e. F, M, L, I and V).

Furthermore, it is expected that there are sequence polymorphisms in the nucleic acid sequence coding for HYP, and it will be appreciated by one skilled in the art that one or more nucleotides in the nucleic acid sequence coding for HYP may vary due to natural allelic variation. Any and all such nucleotide variations and resulting amino acid polymorphisms are within the scope of the invention. It may also be appreciated by one skilled in the art that HYP maybe a member of a family of highly related genes. Nucleotide sequences and corresponding deduced amino acid sequences of any and all such related family members including HYP are within the scope of the invention.

### Example 8

### Production of Antibody Specific to HYP

In order to detect a HYP protein in a sample, antibodies are raised against one or more peptides derived the full length human HYP protein. The chosen regions have a high antigenicity index as predicted by the algorithm of Hopp and Woods (Hopp and Woods, 1981). To increase immunogenicity, antigenic peptides comprise a N- or C-terminal cysteine to allow covalent coupling to a carrier protein (e.g. keyhole limpet hemocyanin, bovine serum albumin). The peptide/carrier complex are injected subcutaneously into animals (e.g. rabbits) and antisera are obtained using standard protocols. Affinity chromatography is used to purify the fraction of immunoglobulins specific to the peptide used to elicit an immune response.

Peptides are synthesized, purified and coupled to activated keyhole limpet hemocyanin via sulfhydryl groups. The peptide/carrier complex is mixed with complete Freund's adjuvant and injected subcutaneously to rabbits on day 1. On days 21, 35 and 49, the peptide/carrier complex mixed with incomplete Freund's adjuvant is again injected. Blood samples are collected on days 44 and 59 to determine the antiserum titer by ELISA. Animals are exsanguinate on day 63. Immunoglobulins are precipitated from pooled antisera and purified by peptide affinity chromatography according to standard protocols (Affinity Bioreagents, Golden, Co.).

### Example 9

### Hyphen-CNK Heterodimerization

Biophysical crystal analysis revealed the formation of a heterodimer of Drosophila Hyphen with a human CNK2 SAM domain. To gain insight into the binding mode of the Hyphen/CNK complex, full-length Drosophila Hyphen and the SAM domain of human CNK2 were separately expressed in bacteria. When combined in vitro, the two proteins co-associated with high affinity and formed a heterodimer as assessed by analytical centrifugation. Crystals of the heterodimer were then obtained and X-ray diffraction analysis revealed its three-dimensional structure. As predicted, a typical interaction between the two SAM explained the heterodimer formation and also identified specific residues enabling the association.

Although the present invention has been described hereinabove by way of specific embodiments thereof, it can be modified, without departing from the spirit and nature of the subject invention as defined in the appended claims.

### REFERENCES

1. Anselmo, A. N., Bumeister, R., Thomas, J. M., and White, M. A. 2002. Critical contribution of linker proteins to Raf kinase activation. J. Biol. Chem. 277: 5940-5943.
2. Baker, D. A., Mille-Baker, B., Wainwright, S. M., Ish-Horowicz, D., and Dibb, N. J. 2001. Mae mediates MAP kinase phosphorylation of Ets transcription factors in Drosophila. Nature 411: 330-334.
3. Bumeister, R., Rosse, C., Anselmo, A., Camonis, J., and White, M. A. 2004. CNK2 couples NGF signal propagation to multiple regulatory cascades driving cell differentiation. Curr. Biol. 14: 439-445.
4. Chong, H., Vikis, H. G., and Guan, K. L. 2003. Mechanisms of regulating the Raf kinase family. Cell. Signal. 15: 463-469.
5. Davies, H., Bignell, G. R., Cox, C., Stephens, P., Edkins, S., Clegg, S., Teague, J., Woffendin, H., Garnett, M. J., Bottomley, W., et al. 2002. Mutations of the BRAF gene in human cancer. Nature 417: 949-954.
6. Dhillon, A. S. and Kolch, W. 2002. Untying the regulation of the Raf-1 kinase. Arch. Biochem. Biophys. 404: 3-9.
7. Dhillon, A. S., Meikle, S., Yazici, Z., Eulitz, M., and Kolch, W. 2002. Regulation of Raf-1 activation and signalling by dephosphorylation. EMBO J. 21: 64-71.
8. Douziech, M., Roy, F., Laberge, G., Lefrancois, M., Armengod, A. V., and Therrien, M. 2003. Bimodal regulation of RAF by CNK in Drosophila. EMBO J. 22: 5068-5078.
9. Jaumot, M. and Hancock, J. F. 2001. Protein phosphatases 1 and 2A promote Raf-1 activation by regulating 14-3-3 interactions. Oncogene 20: 3949-3958.
10. Kolch, W. 2000. Meaningful relationships: the regulation of the Ras/Raf/MEK/ERK pathway by protein interactions. Biochem J. 351: Pt 2:289-305.
11. Kornfeld, K., Hom, D. B., and Horvitz, H. R. 1995. The ksr-1 gene encodes a novel protein kinase involved in Ras-mediated signaling in C. elegans. Cell 83: 903-13.
12. Laberge, G., Douziech, M., and Therrien, M. 2005. Src42 binding activity regulates Drosophila RAF by a novel CNK-dependent derepression mechanism. EMBO J. 24: 487-498.
13. Lanigan, T. M., Liu, A., Huang, Y. Z., Mei, L., Margolis, B., and Guan, K. L. 2003. Human homologue of Drosophila CNK interacts with Ras effector proteins Raf and Rlf. FASEB J. 17: 2048-2060.
14. Light, Y., Paterson, H., and Marais, R. 2002. 14-3-3 antagonizes Ras-mediated Raf-1 recruitment to the plasma membrane to maintain signaling fidelity. Mol. Cell. Biol. 22: 4984-4996.
15. Malumbres, M. and Barbacid, M. 2003. RAS oncogenes: the first 30 years. Nat. Rev. Cancer 3: 459-465.
16. Morrison, D. K. and Cutler, R. E. 1997. The complexity of Raf-1 regulation. Curr. Opin. Cell Biol. 9: 174-179.
17. Morrison, D. K. and Davis, R. J. 2003. Regulation of MAP kinase signaling modules by scaffold proteins in mammals. (2003). Annu. Rev. Cell. Dev. Biol. 19: 91-118.
18. Pearson, G., Robinson, F., Beers Gibson, T., Xu, B. E., Karandikar, M., Berman, K., and Cobb, M. H. 2001. Mitogen-activated protein (MAP) kinase pathways: regulation and physiological functions. Endocr. Rev. 22: 153-183.
19. Qiao, F. and Bowie, J. U. 2005. The many faces of SAM. Sci STKE 2005: re7.
20. Pirrotta, V. 1988. Vectors for P-element transformation in Drosophila. In Vectors, a survey of molecular cloning vectors and their uses (ed, R. L. Rodriguez and D. T. Denhardt), pp. 437-456. Butterworths, Boston, Mass.
21. Rebay, I. and Rubin, G. M. 1995. Yan functions as a general inhibitor of differentiation and is negatively regulated by activation of the Ras1/MAPK pathway. Cell 81: 857-866.
22. Rocheleau, C. E., Ronnlund, A., Tuck, S., and Sundaram, M. V. 2005. Caenorhabditis elegans CNK-1 promotes Raf activation but is not essential for Ras/Raf signaling. Proc. Natl. Acad. Sci. USA 102: 11757-11762.
23. Roy, F., Laberge, G., Douziech, M., Ferland-McCollough, D., and Therrien, M. 2002. KSR is a scaffold required for activation of the ERK/MAPK module. Genes Dev. 16: 427-438.
24. Slupsky, C. M., Gentile, L. N., Donaldson, L. W., Mackereth, C. D., Seidel, J. J., Graves, B. J., and Mcintosh, L. P. 1998. Structure of the Ets-1 pointed domain and mitogen-activated protein kinase phosphorylation site. Proc. Natl. Acad. Sci. USA 95: 12129-12134.
25. Stapleton, D., Balan, I., Pawson, T., and Sicheri, F. 1999. The crystal structure of an Eph receptor SAM domain reveals a mechanism for modular dimerization. Nat. Struct. Biol. 6: 44-49.
26. Sundaram, M. and Han, M. 1995. The C. elegans ksr-1 gene encodes a novel Raf-related kinase involved in Ras-mediated signal transduction. Cell 83: 889-901.
27. Therrien, M., Chang, H. C., Solomon, N. M., Karim, F. D., Wassarman, D. A., and Rubin, G. M. 1995. KSR, a novel protein kinase required for RAS signal transduction. Cell 83: 879-888.
28. Therrien, M., Michaud, N. R., Rubin, G. M., and Morrison, D. K. 1996. KSR modulates signal propagation within the MAPK cascade. Genes Dev. 10: 2684-2695.
29. Therrien, M., Wong, A. M., and Rubin, G. M. 1998. CNK, a RAF-binding multidomain protein required for RAS signaling. Cell 95: 343-353.
30. Wan, P. T., Garnett, M. J., Roe, S. M., Lee, S., Niculescu-Duvaz, D., Good, V. M., Jones, C. M., Marshall, C. J., Springer, C. J., Barford, D., and Marais, R. Cancer Genome Project. 2004. Mechanism of activation of the RAF-ERK signaling pathway by oncogenic mutations of B-RAF. Cell 116: 855-867.
31. Wellbrock, C., Karasarides, M., and Marais, R. 2004. The RAF proteins take centre stage. Nat Rev Mol. Cell. Biol. 5: 875-885.
32. Zhang, B. H. and Guan, K. L. 2000. Activation of B-Raf kinase requires phosphorylation of the conserved residues Thr598 and Ser601. EMBO J. 19: 5429-5439.
33. Ziogas, A., Moelling, K., and Radziwill. G. 2005. CNK1 is a scaffold protein that regulates Src-mediated Raf-1 activation. J. Biol. Chem. 280: 24205-24211.
34. Douziech et al. 2006

What is claimed is:

1. A purified polypeptide comprising a SAM domain, the sequence of said SAM domain being as set forth in SEQ ID NO: 33.

2. The polypeptide of claim 1, the sequence of said SAM domain being as set forth in SEQ ID NO: 34.

3. The polypeptide of claim 1, the sequence of said SAM domain being as set forth in SEQ ID NO: 35.

4. The polypeptide of claim 1, the sequence of said SAM domain being as set forth in SEQ ID NO: 36.

5. The polypeptide of claim 1, the sequence of said SAM domain being as set forth in SEQ ID NO: 37.

6. The polypeptide of claim 1, the sequence of said SAM domain being as set forth in SEQ ID NO: 17.

7. The polypeptide of claim 1, the sequence of said SAM domain being as set forth in SEQ ID NO: 18.

8. The polypeptide of claim 1, the sequence of which being as set forth in SEQ ID NO: 1.

9. The polypeptide of claim 1, the sequence of which being as set forth in SEQ ID NO: 2.

10. A purified antibody that specifically binds to the polypeptide of claim 5.

11. A purified antibody that specifically binds to the polypeptide of claim 6.

12. A purified antibody that specifically binds to the polypeptide of claim 7.

13. A method of determining whether a biological sample contains the polypeptide defined in claim 5, comprising contacting the sample with a purified ligand that specifically binds to the polypeptide, and determining whether the ligand specifically binds to the sample, the binding being an indication that the sample contains the polypeptide.

14. The method of claim 13 wherein the ligand is a purified antibody that specifically binds to the polypeptide.

15. A method of purifying the polypeptide defined in claim 5 from a biological sample containing the polypeptide, said method comprising: contacting the biological sample with a purified ligand that specifically binds to the polypeptide, the ligand being bound to a solid support, to produce a ligand-polypeptide complex, separating the complex from the remainder of the sample, and releasing the polypeptide from the ligand thereby obtaining the purified polypeptide.

16. The method of claim 15 wherein the ligand is a purified antibody that specifically binds to the polypeptide.

17. A kit comprising a purified ligand that specifically binds to the polypeptide of claim 5, and instructions to use the ligand for detecting the polypeptide in a biological sample.

18. The kit of claim 17, wherein the ligand is a purified antibody that specifically binds to the polypeptide.

19. A kit comprising a purified ligand that specifically binds to the polypeptide of claim 5, and instructions to use the ligand for purifying the polypeptide from a biological sample.

20. The kit of claim 19, wherein the ligand is a purified antibody that specifically binds to the polypeptide.

21. An isolated polynucleotide encoding the polypeptide defined in claim 5.

22. A probe specifically hybridizable to the polynucleotide of claim 21.

23. A method of modulating RAS-mediated MAPK activation comprising modulating the biological activity of a polypeptide defined in claim 5.

24. The method of claim 23, wherein said modulating is increasing the biological activity of the polypeptide.

25. The method of claim 24, wherein said modulating is inhibiting the biological activity of the polypeptide.

26. The method of claim 25, wherein said step of inhibiting the biological activity of said polypeptide is conducted in vitro.

27. The method of claim 25, wherein said step of inhibiting the biological activity of said polypeptide is conducted in vivo.

28. The method of claim 25, wherein said step of inhibiting the biological activity of said polypeptide comprises administering a short interfering RNA molecule (siRNA) that decreases expression of said polypeptide.

29. The method of claim 25, wherein said step of inhibiting the biological activity of said polypeptide comprises administering a purified antibody that specifically binds to said polypeptide.

30. A short interfering RNA molecule (siRNA) that decreases the expression of a polypeptide by RNA interference, said polypeptide comprising a SAM domain, the sequence of said SAM domain being as set forth in SEQ ID NO: 37, comprising a sense region and an antisense region, wherein said antisense region comprises a sequence complementary to an RNA sequence encoding said polypeptide and the sense region comprises a sequence complementary to the antisense of said RNA sequence encoding said polypeptide.

31. The siRNA of claim 30, wherein said siRNA molecule is assembled from two nucleic acid fragments, wherein one fragment comprises the sense region and the second fragment comprises the antisense region of said siRNA molecule.

32. The siRNA of claim 30, wherein said sense region and said antisense region are covalently connected via a linker molecule.

33. The siRNA of claim 30, wherein said linker molecule is a polynucleotide linker molecule.

34. The siRNA molecule of claims 30, wherein said sense region comprises a 3′-terminal overhang of 1 to 5 nucleotides in length and said antisense region comprises a 3′-terminal overhang of 1 to 5 nucleotides in length.

35. The siRNA molecule of claims 30, wherein said sense and antisense regions comprise at least one nucleotide that is chemically modified in at least one of sugar, base, or backbone moiety.

36. The siRNA molecule of claims 30, comprising a double stranded region of about 10 to 28 nucleotides in length.

37. The siRNA molecule of claim 36, wherein said siRNA molecule is linked to at least one receptor binding ligand.

38. The siRNA molecule of claim 37, wherein said receptor binding ligand is attached to the 5′-end, the 3′ end or both ends of the sense or antisense region of the siRNA molecule.

39. A method to identify a modulator of the polypeptide defined in claim 5, comprising:
a) contacting a candidate compound with cells expressing said polypeptide; and
b) assessing said cells for an alteration in a biological activity of said polypeptide;
wherein a modulator of said polypeptide is identified when said biological activity is altered in the presence of the candidate compound as compared to in the absence thereof.

40. The method of claim 39, wherein said modulator is an inhibitor and wherein an inhibitor of said polypeptide is identified when said biological activity is decreased in the presence of a candidate compound as compared to in the absence thereof.

41. The method of claim 40, wherein said biological activity is MEK activation.

42. A method of inhibiting Ras-dependent CNK RAF activation comprising inhibiting KSR activity.


SEQUENCE LISTING (42 sequences)

SEQ ID NO: 1 - protein, 202 aa, Homo sapiens
MFTELRSKLSPPRGRAGAVRAGFGERRDVDATAHFSFCRTLLEHTVSAESIPCHLPRTPGTSLTWHDSRSQRAASSRPIKLLQQPGTDTPQGRLYSDHYGLYHTSPSLGGLTRPVVLWSQQDVCKWLKKHCPHNYLVYVEAFSQHAITGRALLRLNAEKLQRMGLAQEAQRQEVLQQVLRLQVREEGRSLQLLSQASFGKMS

SEQ ID NO: 2 - protein, 201 aa, Homo sapiens
MAVEALHCGLNPRGIDHPAHAEGIKLQIEGEGVESQSIKNKNFQKVPDQKGTPKRLQAEAETAKSATVKLSKPVALWTQQDVCKWLKKHCPNQYQIYSESFKQHDITGRALLRLTDKKLERMGIAQENLRQHILQQVLQLKVREEVRNLQLLTQGTLLLPDGWMDGEIRRKTTLLLGQTGVRENLLLFLHRISIIENSIQI

SEQ ID NO: 3 - protein, 199 aa, Pan troglodytes
MFTELRSKLSPPRGRAGAVRAGFGERRDVDDFSFCWTLLEHTVSAESIPCHLPRTPGTSLTWHDSRSQRAASSRPIKLLQQPGTDTPQGRLYSDHYGLYHTSPSLGGLTRPVVLWSQQDVCKWLKKHCPHNYLVYVEAFSQHAITGRALLRLNAEKLQRMGLAQEAQRQEVLQQVLRLQVREEGRSLQLLSQASFGKMS

SEQ ID NO: 4 - protein, 201 aa, Pan troglodytes
MAVEALHCGLNPRGIDHPAHAEGIKLQIEGEGVESQSIKNKNFQKVPDQKGTPKRLQAEAETAKSATVKLSKPVALWTQQDVCKWLKKHCPNQYQIYSESFKQHDITGRALLRLTDKKLERMGIAQENLRQHILQQVLQLKVREEVRNLQLLTQGTLLLPDGWMDGEIRRKNTLLLGQTGVRENLLLFLHRISIIENSIQI

SEQ ID NO: 5 - protein, 253 aa, Canis familiaris
MHDDSISECAFARGAVGVLWHPHTPGESAQTPGGPPGVRRGVCARPGSLGPMFTELRTKLSPPRGRAGAVRAGFGERRDVDATAHFSFCRTLLEHTVSAESIPCHLPRTPGTSLTWHDSRSQRAAGGRPVKLLQQPGTEAPQGRMYSDHYGLYHTSPSLGGLTRPVVLWSQQDVCKWLKKHCPHNYLVYVEAFSQHAITGRALLRLNGEKLQRMGLAQEAQRQEVLQQVLRLQVREEGRSLQLLSQASFGNAS

SEQ ID NO: 6 - protein, 270 aa, Canis familiaris
MRDVGGSDQGDGSADGAMRMDWSSTVGVELAGPGDRWKGLEFFYSLLALHCGLNPRGIDHSARAEGIKLQIEGEGVESQSIKNKNFQKVLDQKGTPKRLPTEAETAKSATVKLSKPVALWTQQDVCKWLKKHCPNQYQIYSESFKQHDITGRALLRLTDKKLERMGIAQENLRQHILQQVLQLKVREEVRNLQLLTQGILLLPCGWMEGGEDGKKETFTTMTKGSQGETIFVNSDWPSFDAVNNSQHQGVMLPRDSPCGHGEAVTHPEGY

SEQ ID NO: 7 - protein, 247 aa, Bos taurus
MGGHISARRRHPSELPERARLSGLSGRAALRPARGVCTGGALAGPMFTELRTKPSPPRGRAGAVRSGFGERRDVDATAHFNFCRSLLDHTVSAENIPCPLPRTPGTSLTWHDSRSQRAAGGRPVKLLQQPGTEAPQGRLYSDHYGLYHTSPSLGGLTRPVVLWSQQDVCKWLKKHCPHNYLVYVEAFSQHAITGRALLRLNAEKLQRMGLAQEAQRQEVLQQLLHLQVREEGRSLQLLSQASFGNMS

SEQ ID NO: 8 - protein, 105 aa, Bos Taurus
SATVKLSKPVALWTQQDVCKWLKKHCPNQYQIYSESFKQHDITGRALLRLTDKKLERMGIAQENLRQLILQQVLQLKVREEVRNLQLLTQGTLLLPSGWMRGEAG

SEQ ID NO: 9 - protein, 202 aa, Mus musculus
MFTELRSKLSPPRARAGAVRPGFGERPDVDASAHFSFCQTLLEHTVSAENIPCHLPRTPGTSLTWHDSRSQRASSSRPIKLLQQPGSEIPQARLYSDHYGLYHTSPSLGGLTRPVVLWSQQDVCKWLKKHCPHNYLVYVEAFSQHAITGRALLRLNADKLQRMGLTQEAQRQEVLQQVLHLQVREEGRSLKLLSQASFGNMS

SEQ ID NO: 10 - protein, 161 aa, Mus musculus
MAVEALHCGLNPRGIDHSAHADGIKLQIEGEGVESQSIKNRTFQKVPDQKGTPKRLQGEAETAKSATVKLSKPVALWTQQDVCKWLKKHCPNQYQLYSESFKQHDITGRALLRLTDKKLERMGIAQENQRQHILQQVLQLKVREEVRNLQLLTQASVECSP

SEQ ID NO: 11 - protein, 352 aa, Rattus norvegicus
MRRSQKLSSPVQERFGAGKAAKLLSSAIITTSNLPPRGPRLPTAKAVQRVAREAGPRSPTQVRKDAFCSGGHPQHTQPGAPPIHLRAPGARLAPRAGRQVLCRADSPAAGTGLGSAGSGGWGRGARCPRATLAAWCSARGVCPSRASAGSMFTELRSKLSPPRARAGAVRTGFGERPDVDASAHFSFCQTLLEHTVSAENIPCHLPRTPGTSLTWHDSRSQRASSSRPIKLLQQPGSETPQARLYSDHYGLYHTSPSLGGLTRPVVLWSQQDVCKWLKKHCPHNYLVYVEAFSQHAITGRALLRLNADKLQRMGLTQEAQRQEVLQQVLHLQVREEGRSLQLLSQASFGNMS

SEQ ID NO: 12 - protein, 161 aa, Rattus norvegicus
MAVEALHCGLNPQSIDHPAHADGIKLQIEGEGVESQSIKNRNFQKVSDQKGTPKRLQGEAETAKSATVKLSKPVALWTQQDVCKWLKKHCPNQYHIYSESFKQHDITGRALLRLTDKKLERMGVAQENQRQHILQQVLQLKVREEVRNLQLLTQASTDCSP

SEQ ID NO: 13 - protein, 197 aa, Gallus gallus
MHQGRPSLCCVASIRGSQGAPEPAAAAAAHFSFCRSLLEHTVSAENLSYRLQRSPGTSLTWHDGRSQRGEGSRAIKLLRQPGTEGSQSRGCDHYGIYHTSPTLGSLTKPVVLWSQQDVCKWLKKHCPHNYLIYVEAFSHHAITGRALLRLNGEKLQRMGIAHEAQRQEVLQQVLQLQVREEVRNLQLLSQASFGNVS

SEQ ID NO: 14 - protein, 382 aa, Gallus gallus
MEKLRMPGLLEREGKMAAIHCNLNPNGIDHPVPTEGINLRLEGEGAESPSAKNSSAPKGPESKETPKRQQVEPEISKVVVPPLLALSLVLRLWCAFLKECSQPRLRAIHCNLNPNGIDHPVPTEGINLRLEGEGAESPSAKNSSAPKGPESKETPKRQQVEPEISKNVGERLPNVKAAMFGDAVYYDLGKPEVIQQQSCLLGKRADTDRIKFLKDTRTYSLLKDFWSCYLGIAVKKHCRIVTSRLGKFKEEPFISIAAQGAVAGHLLFIMRLSLLKICECLQQIASGTVKLTKPVALWTQQDVCKWLKKHCPNQYQIYSESFKQHDITGRALLRLTDKKLERMGIAQESLRQHILQQVLQLKVREEVRNLQLLTQAPAESSP

SEQ ID NO: 15 - protein, 106 aa, Drosophila melanogaster
MGEETINSTQNKTRTKTTRPKAVYLWTVSDVLKWYRRHCGEYTQYEQLFAQHDITGRALLRITDSSLQRMGVTDNRDREAIWREIVKQRLKTDIMEIRDMERLNIY

SEQ ID NO: 16 - protein, 102 aa, Caenorhabditis elegans
MTTNVQEVYHVVSHRPIYFWSANDVDQWLRRKRPRFALKYAHFFLRHNVTGRVLVEMSDDDLREIGVETDDERQDLLLEIKKEKLYSDLDEFSKLRTPPTAI

SEQ ID NO: 17 - protein, 70 aa, Homo sapiens
VVLWSQQDVCKWLKKHCPHNYLVYVEAFSQHAITGRALLRLNAEKLQRMGLAQEAQRQEVLQQVLRLQVR

SEQ ID NO: 18 - protein, 70 aa, Homo sapiens
VALWTQQDVCKWLKKHCPNQYQIYSESFKQHDITGRALLRLTDKKLERMGIAQENLRQHILQQVLQLKVR

SEQ ID NO: 19 - protein, 70 aa, Pan troglodytes; identical to SEQ ID NO: 17

SEQ ID NO: 20 - protein, 70 aa, Pan troglodytes; identical to SEQ ID NO: 18

SEQ ID NO: 21 - protein, 70 aa, Canis familiaris
VVLWSQQDVCKWLKKHCPHNYLVYVEAFSQHAITGRALLRLNGEKLQRMGLAQEAQRQEVLQQVLRLQVR

SEQ ID NO: 22 - protein, 70 aa, Canis familiaris; identical to SEQ ID NO: 18

SEQ ID NO: 23 - protein, 70 aa, Bos taurus
VVLWSQQDVCKWLKKHCPHNYLVYVEAFSQHAITGRALLRLNAEKLQRMGLAQEAQRQEVLQQLLHLQVR

SEQ ID NO: 24 - protein, 70 aa, Bos taurus
VALWTQQDVCKWLKKHCPNQYQIYSESFKQHDITGRALLRLTDKKLERMGIAQENLRQLILQQVLQLKVR

SEQ ID NO: 25 - protein, 70 aa, Mus musculus
VVLWSQQDVCKWLKKHCPHNYLVYVEAFSQHAITGRALLRLNADKLQRMGLTQEAQRQEVLQQVLHLQVR

SEQ ID NO: 26 - protein, 70 aa, Mus musculus
VALWTQQDVCKWLKKHCPNQYQLYSESFKQHDITGRALLRLTDKKLERMGIAQENQRQHILQQVLQLKVR

SEQ ID NO: 27 - protein, 70 aa, Rattus norvegicus; identical to SEQ ID NO: 25

SEQ ID NO: 28 - protein, 70 aa, Rattus norvegicus
VALWTQQDVCKWLKKHCPNQYHIYSESFKQHDITGRALLRLTDKKLERMGVAQENQRQHILQQVLQLKVR

SEQ ID NO: 29 - protein, 70 aa, Gallus gallus
VVLWSQQDVCKWLKKHCPHNYLIYVEAFSHHAITGRALLRLNGEKLQRMGIAHEAQRQEVLQQVLQLQVR

SEQ ID NO: 30 - protein, 70 aa, Gallus gallus
VALWTQQDVCKWLKKHCPNQYQIYSESFKQHDITGRALLRLTDKKLERMGIAQESLRQHILQQVLQLKVR

SEQ ID NO: 31 - protein, 69 aa, Drosophila melanogaster
VYLWTVSDVLKWYRRHCGEYTQYEQLFAQHDITGRALLRITDSSLQRMGVTDNRDREAIWREIVKQRLK

SEQ ID NO: 32 - protein, 70 aa, Caenorhabditis elegans
IYFWSANDVDQWLRRKRPRFALKYAHFFLRHNVTGRVLVEMSDDDLREIGVETDDERQDLLLEIKKEKLY

SEQ ID NO: 33 - protein, 71 aa, Artificial Sequence
  Consensus sequence of SAM domains from various species including nematode
XXXWXXXDVXXWXXXXXXXXXXXYXXXFXXHXXTGRXLXXXXXXXLXXXGXXXXXXRXXXXXXXXXXXXXX

SEQ ID NO: 34 - protein, 71 aa, Artificial Sequence
  Consensus sequence of SAM domains from two human hyp
VXLWXQQDVCKWLKKHCPXXYXXYXEXFXQHXITGRALLRLXXXKLXRMGXAQEXXRQXXLQQVLXLXVRE

SEQ ID NO: 35 - protein, 71 aa, Artificial Sequence
  Consensus sequence of SAM domains from various species excluding nematode
VXLWXXXDVXKWXXXHCXXXXXXYXXXFXXHXITGRALLRLXXXXLXRMGXXXXXXRXXXXXXXXXXXXXX

SEQ ID NO: 36 - protein, 71 aa, Artificial Sequence
  Consensus sequence derived from SAM domains of various species excluding nematode and fly
VXLWXQQDVCKWLKKHCPXXYXXYXEXFXXHXITGRALLRLXXXKLXRMGXXXEXXRQXXLQQVLXLXVRE

SEQ ID NO: 37 - protein, 71 aa, Artificial Sequence
  Consensus sequence derived from SAM domains of various species excluding nematode, fly and chicken
VXLWXQQDVCKWLKKHCPXXYXXYXEXFXQHXITGRALLRLXXXKLXRMGXXQEXXRQXXLQQVLXLXVRE

SEQ ID NO: 38 - protein, 14 aa, Artificial Sequence
  V5 epitope
GKPIPNPLLGLDST

SEQ ID NO: 39 - protein, 7 aa, Artificial Sequence
  PYO epitope
MEYMPME

SEQ ID NO: 40 - protein, 6 aa, Artificial Sequence
  AU5 epitope
TDFYLK

SEQ ID NO: 41 - protein, 108 aa, Anopheles gambiae
TVHASPQTLHYSQLKMARPKPVYMWTVADTLKWLWRHCNERCVKYQELFQRHEITGRALLRITDEQTLYRMGIHDEKDRGDILREIIKQRLKTDIMEIRDMELMNNVY

SEQ ID NO: 42 - protein, 107 aa, Anopheles melas
MVEEVVNSTNKPKNKTARPRPVYLWNVLDVQKWLRRHCSDYYQLYHEKFLYHDITGRVLLRINENILLRLGIDNEQHRLDIWREIMKLHLKTDMLEIKDIERLLDIY